(19) United States
(12) Patent Application Publication
(10) Pub. No.: US 2006/0184040 A1
(43) Pub. Date: Aug. 17, 2006

(54) Apparatus, system and method for optically analyzing a substrate

(76) Inventors: Kurtis Pierce Keller, Hillsborough, NC (US); Caroline Kelly Green, Chapel Hill, NC (US); Andrei State, Chapel Hill, NC (US); Adam Wax, Chapel Hill, NC (US)

Correspondence Address: MOORE & VAN ALLEN PLLC, P.O. BOX 13706, Research Triangle Park, NC, 27709, US

(21) Appl. No.: 11/295,857

(22) Filed: Dec. 7, 2005

Related U.S. Application Data
(60) Provisional application No. 60/634,510, filed on Dec. 9, 2004.

(51) Int. Cl. A61B 6/00

(52) U.S. Cl. 600/476; 600/473

(57) ABSTRACT

An apparatus for optically analyzing a substrate. The apparatus includes: (a) a light source for directing light onto the substrate; (b) optics for creating an optical path from light reflected from the substrate; and (c) a multiple wavelength imaging optical subsystem positioned in the optical path. The multiple wavelength imaging optical subsystem includes: (i) one or more filters which are capable of one or both of: (1) being alternatively or sequentially interposed in the optical path to extract one or more of wavelengths or wavelength bands of interest; or (2) having their wavelength selectivity adjusted to extract one or more wavelengths or wavelength bands of interest; and (ii) one or more imaging devices positioned to image the extracted wavelengths or wavelength bands of interest from the one or more filters; (d) an imaging device positioned in the optical path. Also a method is included, making use of the apparatus for analysis of a substrate.

The present application is based on and claims priority to Provisional U.S. patent application Ser. No. 60/634,510, entitled “Optical Detection and Classification of Pre-Cancers and Cancers via Endoscopes, Colposcopes, and Optical Systems,” filed on Dec. 9, 2004 by Kurtis Keller et al.

### 1 Field of the Invention

The present invention relates to an apparatus, system and method for optically analyzing a substrate. The invention also relates to a multiple wavelength-imaging optical subsystem (MWIOS) for use in an apparatus of the invention. Further, the invention relates to optical scopes, such as diagnostic scopes, which include the MWIOS, and to optical scope systems which may also include light emitting, collecting, and analysis capability for analysis and/or diagnosis of tissue abnormalities, particularly human tissue abnormalities. The invention also relates to methods of using the apparatus, system and method of the invention in the analysis of a substrate, such as a tissue substrate, particularly a human tissue substrate, and for use in analysis and/or diagnosis of tissue abnormalities, particularly human tissue abnormalities.

### 2 BACKGROUND OF THE INVENTION

According to the American Cancer Society, 1,372,910 Americans will be diagnosed with cancer in 2005, not including basal and squamous cell skin cancers, with which more than a million people will be diagnosed in the U.S. this year. Approximately 570,280 people in the U.S. will die of cancer this year (2005); this is equivalent to the deaths of 1,562 people per day. In terms of specific cancers, 16,380 women will be diagnosed in the U.S. this year (2005) with cancers of the cervix, vulva and vagina, while 149,280 Americans will be diagnosed with cancers of the colon, rectum, anus, anal canal and anorectum. The number of lives claimed by these diseases in the U.S. is estimated to be 5,390 and 56,910, respectively.

Due in large part to a rise in early detection techniques, the five-year survival rate for all cancers in the U.S. is increasing, from 50% in 1974-1976, to 64% between 1995 and 2000. Furthermore, the number of deaths from cervical cancer dropped by 74% between 1955 and 1992, again primarily due to early detection, and the pervasiveness of the Pap smear test [NCI04]. Today, the five-year relative survival rate for invasive cervical cancer discovered during its earliest stage is nearly 100% [ACS04]. However, trends in survival rates for colorectal cancers are not as encouraging. While the five-year survival rate for colorectal cancers caught in the earliest stages is 90%, only an estimated 39% of cases are discovered before more permanent damage is done. A lack of symptoms at early stages is chiefly responsible for this low percentage, suggesting that improved screening capabilities will result in a reduction of morbidity and mortality associated with these conditions.

There is a need in the art for improved methods of detecting tissue lesions, like cancerous and pre-cancerous lesions. With respect to cervical cancer, the Papanicolaou (Pap) test, the dominant standard in cervical cancer screening, has been an invaluable tool in early detection of this disease, but it has many limitations. The accuracy of the Pap test is reported to be difficult to quantify for various reasons; a meta-analysis done by Fahey, et al. in 1995 asserted that the true sensitivity and specificity of the test lay somewhere in the ranges 11-99% and 14-97% respectively [FAHE95]. When abnormal Pap test results are received, a subject will then typically undergo a colposcopic examination for further analysis.

However, colposcopy has been reported to have significant shortcomings as well. Since the colposcope is in essence a low-power microscope, and the earliest pre-cancers are barely visible to the naked eye even under magnification, the physician's ability to recognize pre-cancerous lesions increases to acceptable levels only as he/she becomes more experienced [BUXT91]. Mitchell, et al. performed a meta-analysis of colposcopy and reported that when conducted by experts it achieves high sensitivity (the weighted average of several surveys was 96%), but still reaches an average specificity of only 48% [MITC98]. Because of this low specificity, over $6 B is spent annually on biopsy confirmation (the next step after colposcopy) in the U.S. [CANT98]. Thus, Homung, et al. concluded that “there is a strong need for additional diagnostics that could become rapid, ‘online’ procedures implemented by physicians and nurse practitioners. Overall, this would facilitate more sensitive and cost-effective screening and follow-up of pre-malignant lesions” [HORN99]. This need extends to other lesions, such as colorectal cancer, and other epithelium-based cancers to provide an expeditious, scientifically objective method of diagnosis.

### 3 SUMMARY OF THE INVENTION

The invention includes an apparatus for optically analyzing a substrate. The apparatus generally includes a light source for directing light onto the substrate; optics for creating an optical path from light reflected from the substrate; a multiple wavelength imaging optical subsystem (MWIOS) positioned in the optical path; and an imaging device positioned in the optical path. The MWIOS generally includes one or more filters, which can be alternatively or sequentially interposed in the optical path to extract wavelengths or wavelength bands of interest and/or which may have their wavelength selectivity adjusted to extract one or more wavelengths or wavelength bands of interest; and one or more imaging devices positioned to image the extracted wavelengths or wavelength bands of interest from the one or more filters. The apparatus of the invention may also include a means for transmitting image data from the one or more imaging devices, which means can be electronically coupled to a system to permit transmission of data from the imaging device to the system.

The invention also includes an optical scope employing the apparatus of the invention. Typically, the optics of the apparatus are configured to permit a user to view the substrate via the optics. In some cases, the optical scope is configured for medical use. For example, the optical scope may suitably be configured to permit a user to view an organ or anatomical region selected from the group consisting of one or more of airway, bronchi, vagina, cervix, uterus, urinary tract, bladder, esophagus, stomach, duodenum, rectum, sigmoid colon, colon, abdominal cavity, pelvic cavity, thoracic cavity, and epidermis. In some cases the optical scope is configured as an endoscope, such as a colonoscope or colposcope. The optical scope may also be configured as a microscope. In other embodiments, the optical scope is configured as a bronchoscope, colonoscope, colposcope, cystoscope, esophagogastroduodenoscope, hysteroscope, laparoscope, proctosigmoidoscope, or thorascope.

In some cases, the substrate analyzed using the optical scope of the invention is tissue, for instance, human tissue or animal tissue. In a preferred embodiment, the optical scope is configured to capture a full-frame image of an area of the tissue to be examined. For example, in some cases the tissue area to be examined is from about 2 to about 80 mm across at its widest cross-section, preferably from about 5 to about 50 mm across at its widest cross-section. In other embodiments, the area of the tissue to be examined is from about 2 to about 15 mm across at its widest cross-section. The full-frame image may, for example, include a number of pixels between 4,000 and 16,000,000 (or higher). In a preferred embodiment, the full-frame image includes a number of pixels between 4,000 and 16,000,000, and the area to be examined is between 2 mm and 80 mm at its widest cross-section or from about 5 to about 50 mm across at its widest cross-section.

The invention also includes a system which includes the optical scope or apparatus of the invention. In such a system, the optical scope is electronically coupled (e.g., by wire, optical or wireless communications) to a computer system. The computer system may, for example, include typical components of a desktop or laptop computer, such as a computer processor; a means for transmitting image data from the one or more imaging devices to the computer processor; an input device electronically coupled to the computer processor; and/or an output device electronically coupled to the computer processor. Preferably the computer is programmed (e.g., includes code loaded in the computer processor and/or stored on a disk) to permit the user to control one or more system capabilities. The means for transmitting image data from the one or more imaging devices to the computer processor may be any means for electronically transmitting data, for example, a wireless communications device.

Typically the system is programmed and configured to permit electronic storage and/or transmission of data from the images. For example, the system may be programmed and configured to permit electronic transmission of image data. Further, the system is suitably programmed and configured to display analytical results for viewing by the user via the eyepiece of the user interface or via the user console. The system may also be programmed and configured to operate in one or more processing modes, such as continuous-processing mode, thereby acquiring new sets of imagery on which to perform diagnostic analysis in a continuous, uninterrupted manner, and/or single-frame processing mode, in which the user triggers the acquisition and analysis of a single set of images.

In some embodiments, the system of the invention is programmed to analyze optical data from the images. The optical data may, for example, include optical properties, such as the scattering and/or absorbing properties of the substrate. The analysis can be made by measuring the change in the intensity of reflected light over a predetermined spectral range. In certain preferred embodiments, the substrate analyzed comprises tissue, for instance, human tissue or animal tissue, and the computer system is programmed to conduct analysis of the image data from the one or more imaging devices. Preferably the system is configured to image and to analyze a full-frame image of the tissue. For example, in one embodiment, the optical scope of the system is configured as a colposcope; the colposcope is configured to capture a full-frame image of the cervix; and the system is programmed to analyze the full-frame image. Also, for example, in some embodiments, the system is configured to capture a full-frame image of the cervix wherein the image comprises between 4,000 and 16,000,000 pixels. In other embodiments, the image comprises at least 19,000 pixels, 60,000, pixels, at least 100,000 pixels, at least 500,000 pixels, or at least 1,000,000 pixels.

As noted above, the apparatus of the invention includes a light source for illuminating a substrate. The light source may, for example, emit wavelengths comprising wideband white light, including all or portions of the visible, near-infrared and infrared ranges. The light source may, for example, include a physically continuous light source, a ring light, and/or one or more point light sources. The light source may be filtered to remove wavelengths not of interest. For example, wavelengths that can cause image corruption and/or undesirable thermal effects in the images and/or patient discomfort may be removed. Further, the light source may be supplemented with additional light in one or more wavelengths of interest. In certain embodiments, the light source does not include or consist of a xenon light source. Further, in some embodiments, the light source does not include or consist of a UV light source. In some embodiments, the light source includes neither a xenon light source nor a UV light source. The system analyzes wavelengths of interest, which are in some embodiments selected from wavelengths within the visible, near-infrared and infrared bands of light. In some embodiments, the light source emits light comprising one or more wavelengths selected from the group consisting of visible, near-infrared and infrared bands of light. The analyzed wavelengths of interest may in some embodiments include individual wavelengths, combinations of individual wavelengths, or wavelength bands from the visible, near-infrared and infrared ranges.

As noted above, the apparatus of the invention includes an MWIOS. Typically, the MWIOS includes 1, 2, 3, 4, 5, 6 or more filters for isolating wavelengths or bands of interest. Suitable filters may, for example, include interference filters, dichroic filters, multiple-wavelength filters, and/or band-pass filters. The MWIOS also includes 1, 2, 3, 4, 5, 6 or more imaging devices for imaging wavelengths or bands of interest from the filters. In some embodiments the MWIOS includes one imaging device corresponding to each filter.

One or more of the imaging devices may be selected for its capacity to image light from its corresponding filter. The imaging devices may, for example, image a set of one or more continuous or discrete wavelengths or wavelength bands from the visible and/or near-infrared (NIR) ranges. In a preferred embodiment, the MWIOS includes 2, 3, 4, 5, 6 or more filters ordered in a series, where each filter in the series (a) permits a pre-selected set of one or more continuous or discrete wavelengths or wavelength bands to pass through and into an optical path that is directed to and imaged by an imaging device, and (b) reflects light that does not pass through the filter to a next filter in the series; and (c) functions (a) and (b) are performed by all filters in the series in succession until a final filter, which reflects any remaining light to an absorbent substrate.

In certain embodiments, the one or more imaging devices of the MWIOS have the capacity to simultaneously or substantially simultaneously image a set of one or more continuous or discrete wavelengths or wavelength bands selected for spectrally distinctive behavior when interacting with the physical or chemical components of a tissue abnormality.

In certain embodiments, the imaging devices simultaneously or substantially simultaneously image a set of one or more continuous or discrete wavelengths or wavelength bands from the visible, near-infrared, and infrared ranges. In some embodiments, the one or more imaging devices comprise a monochrome imaging device. For example, the one or more imaging devices may include one or more of the following: a CCD-based camera, a CMOS-based camera, an InGaAs-based camera, image intensifier tubes, or mechanically scanning mirror directed to a detector that receives sequentially scanned pixels to form a 2D image. The one or more imaging devices may include a mechanically scanning mirror directed to a detector that receives sequentially scanned pixels to form a 2D image.

In a preferred embodiment, the one or more imaging devices do not comprise a point-source detector. Preferably the system also includes a full-frame imaging device external to the MWIOS for obtaining an unmodified color image of the substrate.

The system includes optics for directing light to various parts of the system. For example, the system may include an entry lens set selected and arranged to focus light reflected from the substrate and to direct such light into the optical path. Various aspects of the system typically require the light path to be split into two or more paths, and the system may include various mechanisms for achieving this purpose.

For example, suitable mechanisms for splitting light in the optical path into two or more output optical paths include beam-splitters, partially-reflecting mirrors, and the like. The optics may include one or more lenses in the optical path before the one or more imaging devices to focus and/or to correct for one or more wavelength-based distortions. The optics may also include one or more polarizers, such as linear or circular polarizers. Further, the optics may include image intensifiers, such as image intensifier tubes. Such image intensifiers may for example be placed in the optical path prior to the imaging devices to enhance imaging of wavelengths that have longer wavelengths or lower intensity than an imaging device can optimally detect. In a typical embodiment, the optical path is infinity focused. The system may also include light absorbing substrate(s) as needed to absorb non-imaged light.

In certain embodiments, the optics include a mechanism for splitting light in the optical path into two or more output optical paths; one of said output paths is directed via the optics to an imaging device (preferably high resolution, color) for recording imagery; and another of said output paths is directed to an eyepiece for viewing by a user. The scope may further include an image display device, viewable by the user, which is electronically coupled to the imaging device. The electronic coupling can be via any means for transmitting a signal, for example, via wire connection, optical connection, wireless connection, or a combination thereof. The electronic coupling will preferably incorporate a computer processor to modify the image prior to transmitting it to the image display device. For example, the modified image may include indicia to identify abnormalities or lesions. In a preferred embodiment, the imaging device has a minimum resolution of 300,000 pixels. In particular, the image display device is placed in an optical path leading to an eyepiece of the optical scope or otherwise positioned to permit the user to view an image displayed on the image display device through the eyepiece.

In some cases, the optics will include a mechanism for alternatively inserting one or more mirrors and beam-splitters into the optical path, or an electronically-alterable reflective-transmissive device. This aspect of the invention serves to alternately include/exclude the MWIOS in the light path. Thus, for example, when one or more of the mirrors or beam-splitters is/are inserted into the optical path, the optical path is separated into at least two separate optical paths comprising (i) a first optical path directed to the MWIOS; and (ii) a second optical path directed through the optics of the system, at least a portion of which reaches an eyepiece for viewing of the image by a user. When another of the mirror(s) or beam-splitter(s) is/are inserted into the optical path, the MWIOS is avoided, and the optical scope functions as a conventional scope.

It will be appreciated that rather than using mirrors and beam-splitters, an electronically-alterable reflective-transmissive device can be included to achieve the same function. For example, an electronically-changeable reflective-transmissive device can be provided having properties which can be changed based on an input signal to alternatively (a) separate the optical path into two separate optical paths: (i) a first optical path directed to an MWIOS; and (ii) a second optical directed through the remaining optics of the system, at least a portion of which reaches an eyepiece for viewing of the image by a user; and (b) reflect the light to avoid the MWIOS such that the optical scope functions as a conventional scope. In various embodiments, the one or more mirrors, beam splitters or partially reflecting optical devices direct greater than about 50, 60, or 70% of the incoming visible light into the MWIOS. In certain embodiments, they direct substantially all of the NIR and IR light into the MWIOS. The mechanism for alternatively inserting a beam splitter or a mirror may, for example, include a rotatable mount, a mirror mounted on the mount, a beam splitter mounted on the mount, and/or a mechanical, electrical or magnetic means for rotating the mount. The means for rotating the mount may, for example, include a dial, lever or switch coupled mechanically or indirectly via electromechanical means to the rotatable mount.

The invention includes a system incorporating an optical scope of the invention electronically coupled to a computer system. For example, the computer system may include a computer processor, a means for transmitting image data from the one or more imaging devices to the computer processor, and one or more peripherals electronically coupled to the computer processor. The peripherals may, for example, include various input and output devices. In a preferred system, the MWIOS is configured to simultaneously image multiple images of the tissue; each image has a separate set of one or more continuous or discrete wavelengths or wavelength bands; and the computer system is programmed to analyze the images to identify spectral abnormalities to identify tissue abnormalities. In another preferred aspect, the MWIOS is configured to image multiple images of the tissue; each image has a separate set of one or more continuous or discrete wavelengths or wavelength bands; and the computer system is programmed to: analyze the images to identify spectral abnormalities to identify one or more tissue abnormalities; and provide output to a user. For example, output may include indicating a diagnosis of the one or more tissue abnormalities, classifying the one or more tissue abnormalities, ruling out one or more diagnoses or classes of abnormnalities, and/or identifying the location of the one or more tissue abnormalities. Preferably, the MWIOS includes 2, 3, 4, 5, 6 or more filters ordered in a series. In such a series arrangement, each filter in the series permits a pre-selected set of one or more continuous or discrete wavelengths or wavelength bands to pass through and into an optical light path that is directed to and imaged by an imaging device and reflects light that does not pass through the filter to a next filter in the series until a final filter, which reflects any remaining light to an absorbent substrate.

In the system aspect of the invention, the computer system may be programmed or include a program or utility stored on a storage medium which instructs the processor to identify variations in spectral signatures across a series of images from the imaging devices. For example, the variations in spectral signatures are associated with tissue abnormalities, such as pre-cancerous and/or cancerous abnormalities, glucose abnormalities, and/or burns. Such abnormalities may, for example, be found in tissue in the gynecological tract, the gastrointestinal tract, the dermis and the epidermis. In a preferred embodiment, the analysis for abnormalities is an analysis of optical characteristics of the epithelial tissue of the cervix or colon.

In one aspect of the invention the processor is programmed (or software is stored on a storage medium) to analyze the substrate based on information from the images about the scattering, absorbing and other such optical properties of the substrate by measuring the change in the intensity of reflected light over a predetermined spectral range. For example, in one embodiment, a change in the intensity of reflected light over a predetermined spectral range that is outside the range of the scattering, absorbing and other such optical properties of normal tissue represents a potential abnormality, or a potential member of a class of abnormalities. As another example, a change in the intensity of reflected light over a predetermined spectral range that is outside the range of the scattering, absorbing and other such optical properties for normal tissue and inside the range of the scattering and absorbing and other such optical properties of a tissue abnormality or class of tissue abnormalities represents a potential abnormality and/or a potential member of a class of abnormalities.

In certain embodiments, a scope of the invention may include an extension including an optical fiber based optical path and associated optics for transmitting light reflected from a substrate, e.g., for use when the substrate to be analyzed is internal, e.g., in the abdomen or in the lumen of the intestine. For example, the extension may include a flexible endoscope. The invention may also include a means for indexing the distance that the extension that has entered into a subject's body.

In a preferred embodiment, the system includes multiple imaging devices and one or more utilities programmed to geometrically register the geometry of the substrate to the pixels of the multiple imaging devices and/or normalize intensity values across multiple images of the substrate from the multiple imaging devices. In certain embodiments, the normalization is accomplished automatically. The normalization may be based on an area of said imagery selected by input from a user. The system may include a utility programmed to extract subsections of said substrate wherein one or both of excessive light intensity or insufficient light intensity prevents imaging of said substrate with sufficient quality to permit the desired analysis. For example, the utility may be programmed to omit such subsections from diagnostic processing and/or to inform the user that said subsections will require re-imaging during one or more of adjusted lighting, filtering, or positioning circumstances. The system may include a utility programmed to identify spectral attributes in image sub-areas characteristic to a tissue abnormality or not characteristic of normal tissue and provide output to a user indicating the location of such image sub-areas. The analysis and output may include classification of the tissue abnormality.

The output may, for example, include a visible color image of said substrate displayed on a user interface; and one or more indicators displayed on the user interface pointing out, circumscribing or highlighting any image sub-areas having spectral attributes characteristic of a tissue abnormality or not characteristic of normal tissue. The output may, for example, include a visible color image of said substrate displayed on a user interface, and one or more indicators displayed on the user interface pointing out, circumscribing or highlighting any image sub-areas having spectral attributes characteristic of a tissue abnormality or not characteristic of normal tissue, and textual or symbolic information displayed on the user interface communicating information relating to classifying the tissue abnormality. The output may include a visible, monochromatic or color image of the tissue substrate, and textual or symbolic information communicating information of relevance to diagnosis or treatment of the tissue abnormality. The system may be programmed to permit a user to provide input causing the system to provide an output image of the substrate which is digitally or optically magnified; an output image of the substrate showing an individual wavelength or wavelength band; and/or an output showing raw spectral data from the substrate.

The invention also includes methods of analyzing substrates using the apparatus of the invention. For example, the invention provides a method of detecting a tissue abnormality which includes emitting light from the light source onto tissue, directing light emitted reflected from the tissue via the optics to the multiple wavelength imaging optical subsystem, and isolating one or more wavelength bands of interest, directing the wavelength bands of interest to the one or more imaging devices, and using the imaging devices to simultaneously capture images of the wavelength bands of interest, transferring image data from the images to a computational system, and analyzing the images for one or more spectral patterns associated with one or more tissue abnormalities, and/or classes of tissue abnormalities.

The analysis may, for example include a determination of the size, location, and stage or classification of any suspected abnormalities. The method may also involve generating a color image of the tissue. The method may involve generating diagnostic data, which can be superimposed onto the color image. The diagnostic data may be superimposed on the color image on a user console or other display means for permitting the user to view the image. The diagnostic data may be superimposed on the color image and viewable through the eyepiece of the optical scope. The analysis may be performed continuously on sets of imagery obtained in real-time or on one or more sets of imagery as triggered by a user. In some aspects, the method involves switching the system between a mode in which the system operates as a conventional optical scope and a mode in which the MWIOS and associated analytical capabilities are activated. The diagnostic data and/or color imagery and/or monochromatic imagery from an examination may be recorded and stored on a storage medium and/or transmitted to another system, e.g., via a network or wireless communication capability.

### 4 BRIEF DESCRIPTION OF THE DRAWINGS

FIG. 1 is a schematic overview of one embodiment of the system of the invention;

FIG. 2 presents a conceptual diagram for the invention's imaging technique;

FIG. 3 shows a schematic overview of a conventional optical scope;

FIG. 4 shows a more detailed view of the dichromatic BS/MC, which can also serve as the enhanced operation enabling and disabling mechanism;

FIG. 5 provides a detailed diagram of the multiple-wavelength imaging optical subsystem of the invention, which will extract electronic images of the selected light wavelengths and send them to the computational system;

FIG. 6 is a flow chart showing the optical path through an embodiment of the invention;

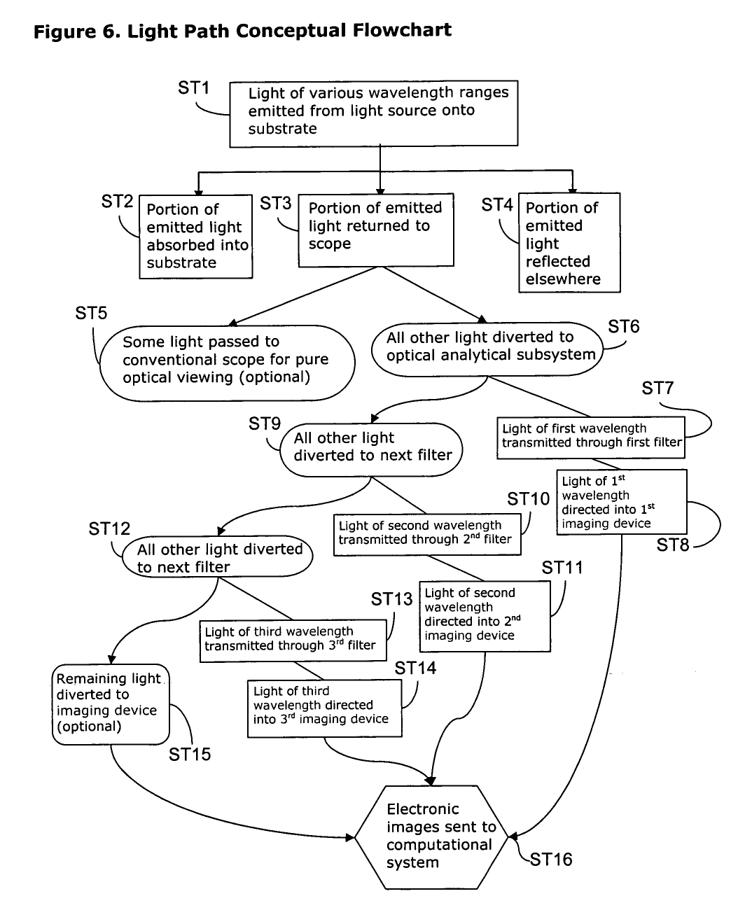

FIG. 7 illustrates the flow of information in the invention's computational system, as it goes from the optical subsystem;

FIG. 8 is a flowchart showing the steps taken in a patient exam according to an embodiment of the invention;

FIG. 9 is a schematic illustration of an embodiment of a system of the invention; and

FIG. 10 is a schematic illustration of another embodiment of a system of the invention.

### 5 Definitions and Abbreviations

“BS/MC” means beam splitter/mirror configuration.

“CCD” means charged-couple device.

“CMOS” means complimentary metal-oxide semi-conductor.

“CRT” means cathode ray tube.

“DRS” means diffuse reflectance spectroscopy.

“Electronically coupled” and the like means coupled via any means capable of transmitting a digital or analog signal. Examples include electrical, optical, radio, and other means, as well as combinations of the foregoing. The signal may be processed or modified in the path of the electronic coupling, e.g., via a computer processor inserted in the path.

“High-resolution” is meant to exclude point-source detectors.

“IEEE” means Institute of Electrical and Electronic Engineers.

“IR” means infrared.

“LED” means light-emitting diode.

“Light path” means the projective path that light travels before making contact with the substrate.

“LCD” means liquid crystal display.

“LCTF” means liquid crystal tunable filter.

“MWIOS” means multiple-wavelength imaging optical subsystem.

“NIR” means near-infrared.

“Optical path” means the reflective path that light reflected from the substrate travels through the system of the invention.

“2D” means two dimensional.

“UV” means ultra-violet.

### 6 DETAILED DESCRIPTION OF THE INVENTION

The invention provides an apparatus, system and method for optically analyzing a substrate. The apparatus, system and method of the invention are useful for recording and analyzing the optical characteristics of the substrate. The substrate is suitably a tissue substrate, for instance, human tissue and/or animal tissue, and the analysis may relate to optical characteristics useful for identifying tissue abnormalities, such as pre-cancerous and cancerous lesions, glucose abnormalities and burn injuries. However, it will be appreciated that the optical scope will be useful in the analysis of other substrates as well, including for example, in vitro tissue samples, manufactured materials, plastics, metals, soils, and other materials.

Embodiments of the system and methods of the invention are further discussed in the ensuing sections. Headings are used for the convenience of the reader only and are not intended to limit the breadth or scope of the invention.

6.1 Optical Scope and System

The invention provides an apparatus, referred to here as an optical scope, and also provides a system comprising the optical scope of the invention along with various information processing capabilities, which will be described in more detail below. Thus, for example, the system of the invention may include an optical scope for obtaining optical information and a system for storing and analyzing optical information obtained using the optical scope.

These two components—optical scope and system components—may be integrated. In other words, the system and optical scope may be provided as one integral unit which includes optical information-gathering capabilities, processing capabilities, data storage capabilities, user interface capabilities, and the like. The optical scope and the associated system components may be provided as a unitary hand-held device. Alternatively, the optical scope may be separate from the data storage and processing aspects of the invention. Data may be transmitted from the optical scope component of the invention to the system components by various transmission means, such as electrical connection, optical connection, infrared connection, radio connection, and the like. In one embodiment, the optical scope and the system are connected wirelessly.

The optical scope of the invention is useful for observing, capturing, recording, and/or transmitting optical data (i.e., data gathered by recording light reflected from a substrate).

It is possible using the teachings of this specification to modify optical scopes used in various medical settings in order to make an optical scope according to the invention. Such an approach may be convenient in some circumstances. For example, in one embodiment, the invention provides a kit for modifying a conventional optical scope to perform the added functions of the invention. Alternatively, the optical scope of the invention may be manufactured de novo as a new article of manufacture.

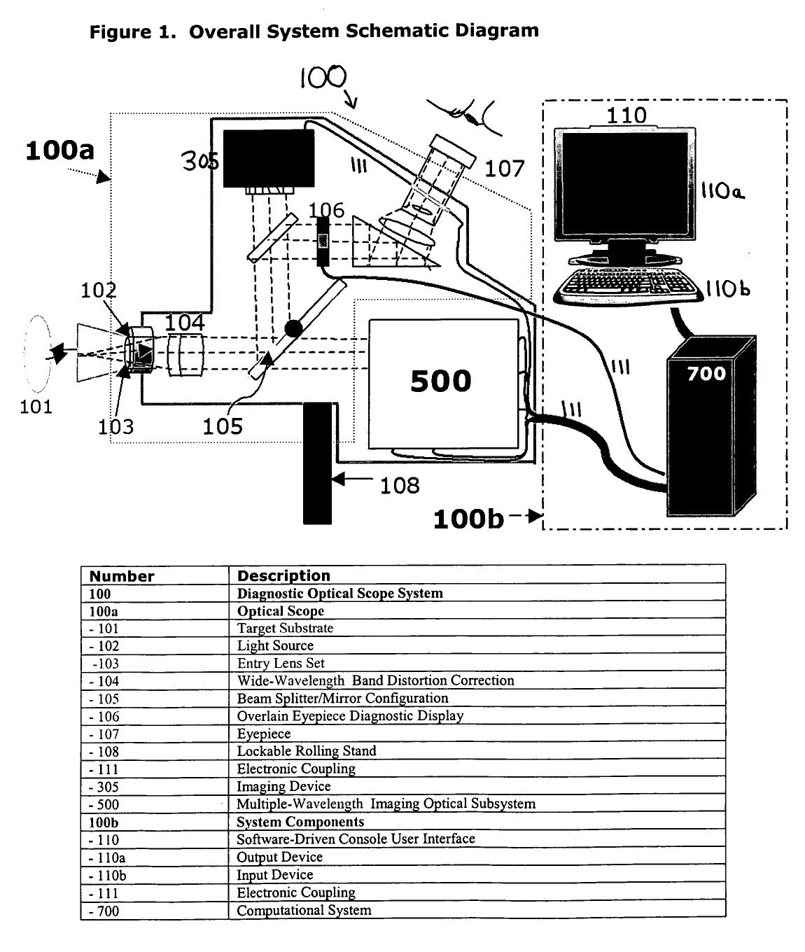

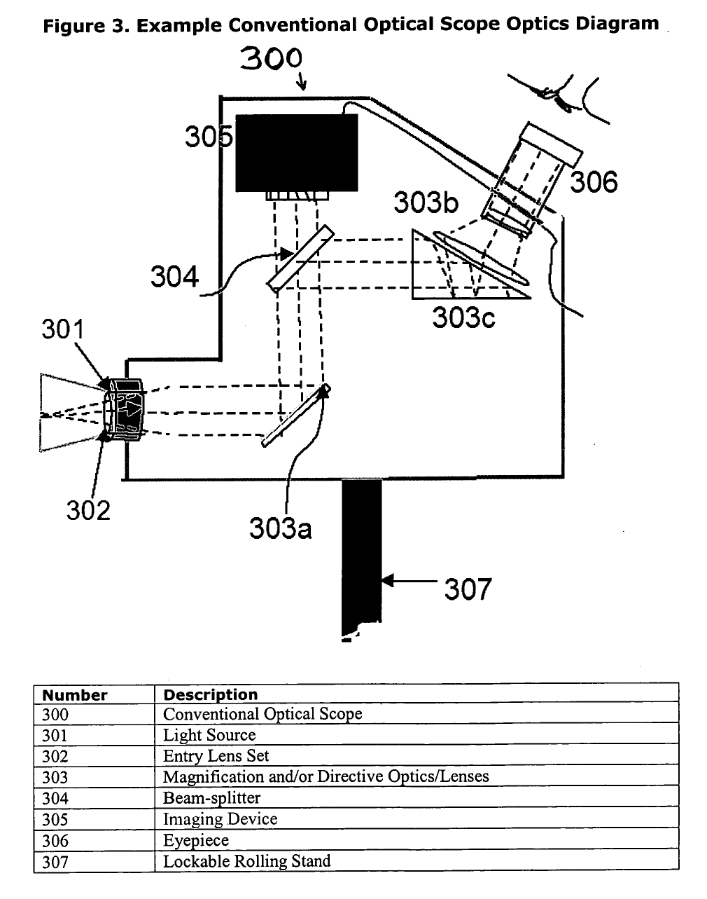

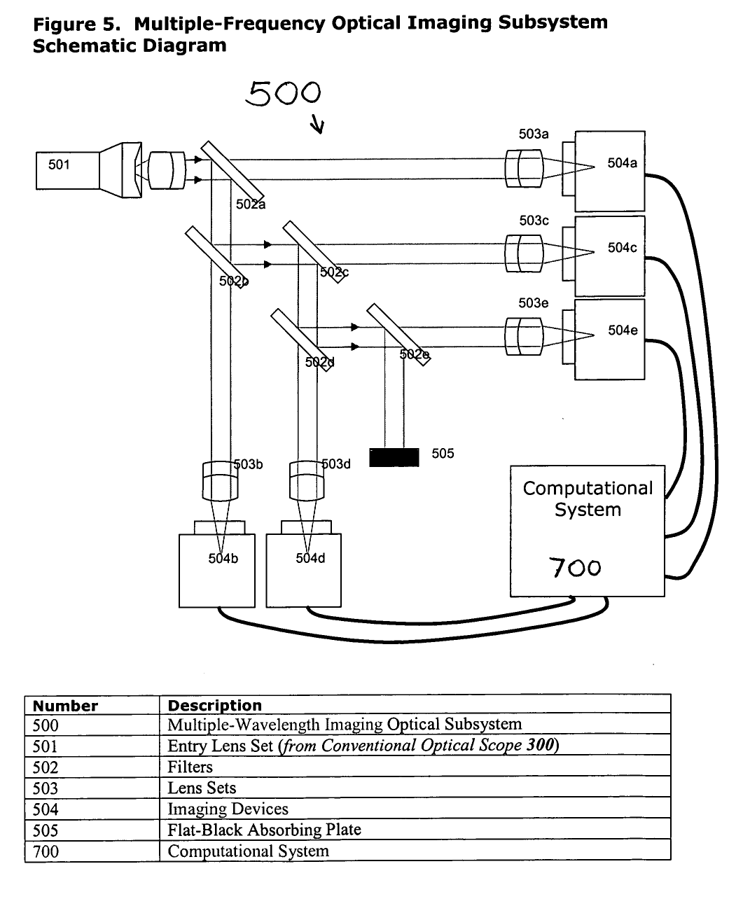

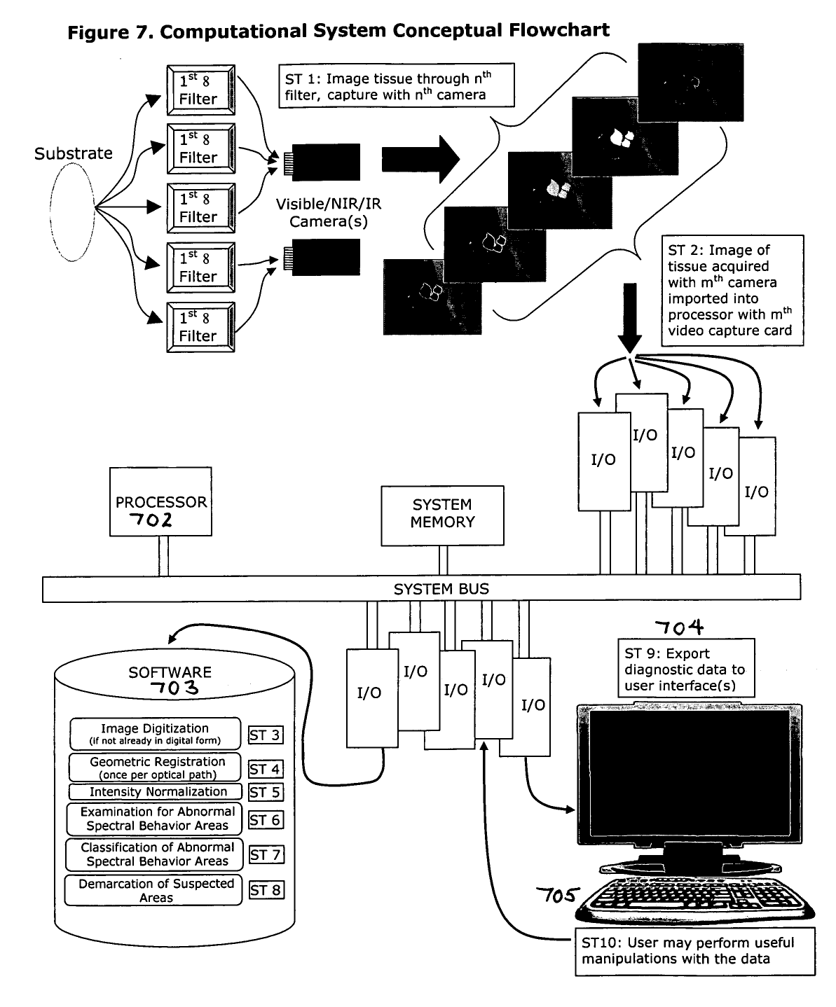

Referring to FIG. 1, the invention provides an optical scope system 100, having an optical scope 100a and system components 100b, for analysis of a substrate 101. The optical scope system 100 generally includes some or all of the following components:
Optical Scope 100a includes:
   Target Substrate 101 to be analyzed
   Light Source 102
   Entry Lens Set 103
   Wide-Wavelength Band Distortion Correction 104
   Beam Splitter-Mirror Configuration 105 (optionally with Enhanced Operation Enabler/Disabler)
   Overlain Eyepiece Diagnostic Display 106
   Eyepiece 107
   Lockable Rolling Stand 108
   Electronic Coupling 111
   Imaging Device 305 (see also, FIG. 3)
   Multiple-Wavelength Imaging Optical Subsystem 500 (see also, FIG. 5)
System Components 100b include:
   Software-Driven Console User Interface 110
   Output Device 110a
   Input Device 110b
   Electronic Coupling 111
   Computational System for Data Analysis 700 (see also, FIG. 7)
   Distance Indexing Mechanism (Optional)

Each of these components is discussed in the ensuing sections.

6.1.1 Light Source

The optical scope 10a of the invention may include a light source 102. The light source 102 may be integral with or separate from the optical scope 100a. The light source 102 functions to emit light, e.g., wideband white light, encompassing all wavelengths of interest. Wavelengths of interest may, for example, include wavelengths within any of the ultra-violet, visible, near-infrared or infrared ranges. The light source 102 emits light onto the surface of the substrate 101 to be examined. This substrate 101, e.g., a tissue substrate, may be illuminated by one or more continuous and/or point light sources. The continuous or point light source(s) may be oriented in any of a variety of patterns, e.g., circular, semicircular or other semi-parallel pattern. These sources can, for example, be centered around the scope's optical path; they can be oriented off-axis; or they may share the optical path of the scope itself.

In some embodiments, the light source 102 is band-filtered in order, for example, to reduce the impact of some heat-producing infrared wavelengths that are not of interest and thereby to improve patient comfort during examination. The light source 102 may also or alternatively be notch-filtered to exclude unnecessary or undesirable wavelengths. For example, NIR and IR radiation can cause excessive amounts of heat, so the portions of those spectra that are not included in the wavelengths of interest may be notch-filtered from the light entering the system of the invention prior to entrance, so as to minimize the amount of heat to be dissipated. The light source 102 may also be pulsed or flashed, in order, for example, to synchronize with the imaging devices 305, 504 (see, FIGS. 3 and 5, respectively) of the invention.

Furthermore, light of specific bands can be injected into or combined with the light from the main light source (white wideband light, for example), to provide additional intensity for some narrowband wavelengths. For example, if in an embodiment of the invention, the wavelength 1000 nm is chosen as a wavelength of interest, and the light source selected for use in said embodiment does not provide adequate intensity thereof, then one or more 1000-nm light-emitting diodes (LEDs) may be used to increase to provide additional intensity. This intensity supplementation can be induced, for example, by filtered wideband light, LEDs, lasers or other light sources.

Examples of light sources suitable for use with the diagnostic endoscope of the invention include xenon arc lamps, quartz halogen lamps, incandescent sources, LEDs, and many others. One preferred embodiment employs a quartz halogen lamp, which tends to be stable, and yields a high output without an excessive amount of heat.

6.1.2 Optical Scope

In some embodiments, the optical scope 100a of the invention includes a configuration of one or more optical, mechanical and electrical components of a conventional optical scope. Conventional optical scope capabilities are useful, among other things, to permit the user to obtain an accurate, unmodified, high-resolution image of the substrate 101 (e.g., tissue area) to be inspected. The term “high-resolution” is meant to exclude point-source detectors. The range of resolutions to be employed in embodiments of the invention will generally range from about 4,000 pixels to about 16,000,000 pixels.

The optical scope 100a may, for example, be configured for medical use. It may be designed for viewing the airway and/or bronchi; the vagina and/or cervix and/or other components of the gynecological tract; inside the uterus; the urinary tract and/or bladder; the esophagus, stomach and/or duodenum; the rectum and/or sigmoid colon; the colon; inside the abdominal cavity and/or pelvis; other components of the gastrointestinal tract; the thoracic cavity; the epidermis; and/or other organs of the body, particularly those covered in epithelial tissue. For example, the optical scope 100a of the invention may be based on a bronchoscope, colonoscope, a colposcope, a cystoscope, a hysteroscope, an esophagogastroduodenoscope, a laparoscope, a proctosigmoidoscope, a thorascope, an endoscope, a microscope, and/or any other appropriate optical scope. The invention includes such scopes modified to perform the functions of the invention, as described herein.

Referring now to FIG. 3, the components of a conventional optical scope 300 typically include:
Light Source 301
Entry Lens Set 302
Magnification and/or Directive Optics/Lenses 303
Beam-splitter (e.g., 50%-50% beam-splitter) 304
Imaging Device (with Lens) 305
Eyepiece 306
Lockable Rolling Stand 307
Fiber Optic Cable for Remote Examination (optional)

Light Source. The optical scope 100a of the invention typically includes one or more components of a conventional optical scope 300, such as a light source 301. Conventional optical scopes 300 use light sources 301 that emit visible wavelengths. Many such light sources 301 are currently available on the market. In some embodiments, the native source on a conventional scope 300 will be insufficient for the analytical and diagnostic applications of the present invention, since the necessary range of wavelengths of the invention will in some embodiments lie outside the visible band. Thus, the conventional visible light source 301 will typically be replaced with a light source that includes wavelengths sufficient to enable the analytical or diagnostic application(s) for which the inventive optical scope 100a is intended.

Elements may be included in the optical path to filter out undesirable wavelengths. For example, those wavelengths not of interest in the NIR and IR ranges can cause a significant amount of heat, so they may be filtered out, through either band-filtering or notch-filtering means. Another example might be UV wavelengths below the lowest wavelength of interest, which are known to cause thermal effects that could cause image corruption.

The light source can be comprised of a single, continuous light source, or several point light sources. In some embodiments where it is desirable for the intensities of certain wavelengths to be increased, the light source may be supplemented with additional single- or multiple-wavelength light sources, such as LEDs.

Entry Lens Set. The optical scope 100a generally includes an entry lens set 302 (also called objective lens set). This lens set 302 gathers and focuses incoming light, which may include light reflected from the substrate 101 being examined, and also serves to direct the light along the appropriate optical path. The entry lens set 302 may include one or more objective lenses; the size and magnification of such lenses is determined by the specific use for which the optical scope 100a is designed.

Focusing and/or Magnification and/or Directive Optics. A conventional optical scope 300 also generally includes focusing and/or magnification and/or directive optics 303. These components typically function to change the focus of, magnify the size of, or modify the direction of the optical path of the image, in addition to other modifications. In a conventional optical scope 300, these optics 303 are often arranged between the entry lens or lens set 302 and the beam-splitter 304, in such a way as to appropriately focus, magnify, reduce, or redirect the optical path. Such optical components may, for example, include lenses (and combinations of lenses) to change the focus or scale of the image; mirrors or angled prisms to change the direction of the image; circular polarizers to reduce specular highlights; and/or other optical components to perform other optical manipulations.

Beam-Splitter. The optical scope 100a generally includes a beam-splitter 105, and also may include a conventional beam-splitter 304, such as a 50-50% beam-splitter or other optical device for dividing an optical path into two or more optical paths. In one embodiment, the beam splitter 304 functions to divide the incoming light into two or images that can be directed to two different targets. As further described below, in a conventional scope set-up, the beam-splitter 304 typically directs a portion of the light to an imaging device 305 for display on an external console and directs the other portion to the eyepiece optics 306. This component may also take the form of a partially-reflecting mirror or similar means.

Imaging Device (with Lens). A conventional optical scope 300 will also typically include an imaging device 305 and an accompanying lens. In other embodiments, the imaging device 305 may be absent. The imaging device 305 may function to record high-resolution, full-color (i.e., wide-band visible-light) images or may record other suitable bands of light. The imaging device 305 serves to record imagery collected during the examination. It may also provide a means to enable a digital console display of the examination imagery. In one embodiment of the invention, the imaging device 305 can provide a color video image that can be optically combined with the wavelength-specific images from the imaging devices 504 of the MWIOS 500, for display purposes.

The imaging device 305 may comprise a color video camera, such as an area camera, a line-scan camera, a focal plane array, or the like. For example, its technology may be based on charged-couple device (CCD), complimentary metal-oxide semiconductor (CMOS) or Indium-Gallium-Arsenide (InG aAs) detectors. In the optical path, the imaging device 305 may, for example, follow the beam-splitter 304 and output its imagery to a user interface (e.g., the eyepiece 306 or user console). The resolution of the imaging device 305 may be, for example, greater than 300,000 pixels.

Eyepiece. A conventional optical scope 300 typically includes an eyepiece 306, which functions to gather and focus the exiting light so that it can be imaged by the human eye. In a conventional optical scope 300, the user can view the tissue area to be inspected through this eyepiece 306. The eyepiece 306 is positioned at the end of the optical path, thereby affording the user a pure optical view of the substrate, and is usually preceded in the path by directive optics 303.

Lockable Rolling Stand. The conventional scope 300 may also include a stand 307 or other mount for the optical scope 100a. This stand 307 functions to provide support for the optical scope 100a. Where the optical scope 100a is used for diagnostic purposes, the stand 307 will be useful to permit the user to correctly position the optical scope 100a during examination. The stand 307 may include lockable rollers, which can be locked to provide stability, e.g., while an exam is in progress. Alternatively, the optical scope 100a may be mounted on any of a variety of moveable mounts, such as pivoting arms mounted on a floor, wall, ceiling, stand, bed or other foundation suitable to the intended use. Where pivoting arms are used, they are preferably lockable.

Fiber Optic Cable for Remote Examination (optional). A conventional optical scope 300 sometimes includes a fiber optic cable in the optical path. Fiber optic cables are well known in the art, and thus, not shown here. Use of a fiber optic cable and associated optics can enable analysis of tissue substrates in locations that are not immediately accessible from the body's exterior (e.g., colon, thorax, etc). This component may, for example, be situated in the optical path between the entry lens set 302 and the first instance of focusing and/or magnification and/or directive optics 303a. This component may take the form of a fiber optic cable, a flexible endoscope, or other such means.

Additional Objective Lens Set for Fiber Optic Cable for Remote Examination. The embodiments of the optical scope 100a that include a fiber optic cable for remote examination may also include an additional set of entry (or objective) lenses 103, 302 suitable for permitting viewing of light emitted from the fiber optic cable. It will be appreciated that various other components of conventional optical scopes not discussed here are well-known in the art and are adaptable for use in optical scopes and systems of the invention.

6.1.3 Wide-Wavelength Band Distortion Correction

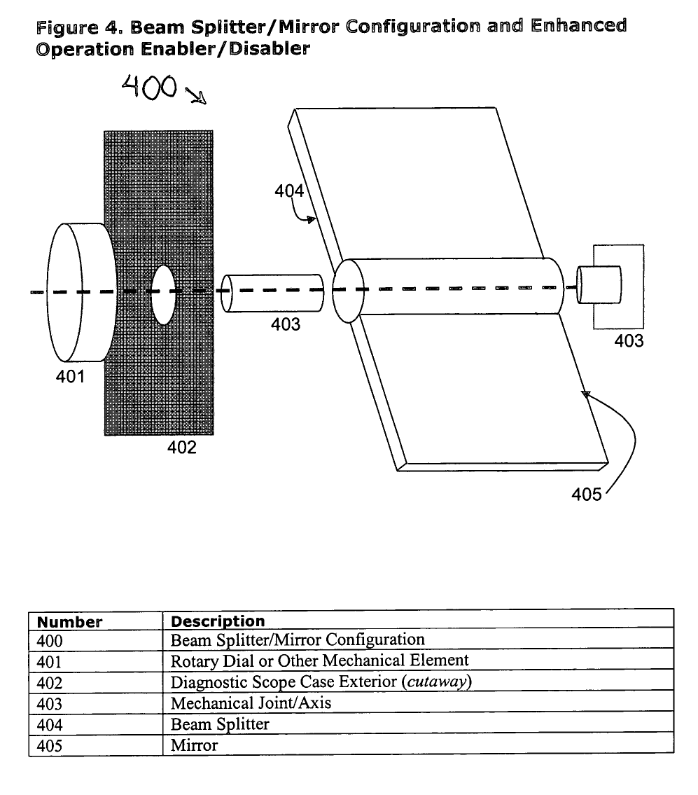

Referring again to FIG. 1, the invention may include an optical component or series thereof 104 to accommodate the wide wavelength-range that is covered, as such wide bands can lead to optical distortion effects. Such a component 104 can, for example, be arranged in the optical path that is after the entry lens 103 and before the BS/MC 105 (shown in more detail in FIG. 4 as BS/MC 400). Optical elements 104 that can be used in this capacity include, for example, one or more achromatic lenses.

6.1.4 Beam Splitter/Mirror Configuration (BS/MC)

As illustrated in FIG. 1, the optical scope system 100 may, in some embodiments, include a beam splitter/mirror configuration 105 (shown in more detail in FIG. 4 as beam splitter/mirror configuration 400), e.g., a dichromatic BS/MC. The BS/MC functions to preserve backward compatibility to the operation of a conventional optical scope. The BS/MC, when present, passes the reflected light either into the (MWIOS) 500 for enhanced operation according to the invention, or to the beam splitter 304 for conventional optical scope operation, depending on its orientation (which can be controlled by the user).

Referring now to FIG. 4, the BS/MC 400 of the invention generally may include:
a mechanical switching component 401, such as a rotary dial, or other mechanical, electronic or magnetic switching component
a diagnostic scope case exterior 402 (shown in cutaway)
a mechanical joint/axis 403, a beam splitter 404 (desirably dichromatic)
a mirror 405
various contact switches, electrical circuitry and control software (not shown).

Thus, in one embodiment, the BS/MC 400 takes the form of a dichromatic beam splitter 404, a mirror 405, and a mechanical joint or axis 403 to which the beam splitter 404 and mirror 405 are rotatably mounted.

The BS/MC 400 may, for example, be controlled by a mechanical switching component 401, such as a rotary dial or other device, which is preferably accessible from the exterior of the optical scope 100a case 402 and is coupled electrically, mechanically or otherwise to permit a user to alternatively place the beam splitter 404 or mirror 405 in the optical path. As an alternative to a mechanical component 401, selection between modes may be accomplished using various computer input devices, such as virtual switch displayed on a monitor which is touch-screen selectable or selectable by a mouse click or other input device.

This component 401 will function to allow the user to select the mode of operation of the system. For example, the components can be mounted and relatively oriented so that, when a user turns the dial (or other device), the beam splitter 404 and the mirror 405 are alternately placed in the optical path.

The relationship of the component 401 with respect to the BS/MC 400 may be mechanical and/or via an electrical circuit and a motion actuator, such as a motor. The controls will preferably include markings, indentations or other indicia for indicating to the user which of the components, beam splitter 404 or mirror 405, is in the optical path or informing the user of the mode of operation. In embodiments in which the component 401 comprises a dial mechanically coupled to an axis 403 on which the beam splitter 404 and mirror 405 are mounted, the dial 401 can also be indexed with the precise angular position points demarcating where the beam splitter 404 and mirror 405 should be in the optical path, e.g., using mechanical detents. It will be appreciated that while this aspect of the invention has been described in terms of a dial, many other configurations will be apparent to one of skill in the art in view of this specification, including for example, various kinds of mechanical, electrical and/or magnetic switches and levers.

The BS/MC 400 can include one or more contact switches (or other such devices) and correspondent electrical circuitry and control software. The switch(es) can be positioned in such a way in order to enact when the user rotates the invention's control dial (or other selector) to position points associated with enabling and disabling the enhanced operation of the invention. When the switch makes contact, it will send an electrical signal through the circuitry to control software that will register it, and commence operation of the lesion detection capability in the computational system 700. The contact switches can be situated so that computational mode of operation is set by the position of the switch so that the system is ready to receive input from the beam-splitter or mirror depending on the position of the component 401 or other input device. Moreover, the system may be programmed to monitor the contact switch or other related signal and to output an indicator, e.g., on the user interface, such as a light or a word or other symbol displayed on a display device, for indicating to the user the mode of operation.

Additionally, in the embodiments of the invention that contain the overlain-eyepiece display 106 as described above in Section 6.1.6, the triggering of the contact switches can also prompt the computational system to enact a motor, such as a servo motor (not shown), for controlling the image display device. The motor can move the image display device into the optical path, blocking the light from the optical scope 100a, and enabling to user to visualize the data.

In one embodiment, the BS/MC 400 directs the incoming light in one of two ways. When the mirror 405 is positioned in the optical path, all incoming light is diverted to the beam splitter 304 to allow for conventional use of the optical scope 100a. When the dichromatic beam splitter 404 is positioned in the optical path, a portion of the light (e.g., 70% of incoming visible light and 100% of NIR and IR light) will be directed into the MWIOS 500, thereby enabling enhanced operation according to the invention. The portion of incoming visible light also may be, for example, less than 50% or greater than 50%. The remainder of the incoming visible light can be directed through the scope optics, in order to provide a raw image of the substrate 101 being examined. Data can be superimposed on the raw image.

The beam splitter 404 can be an optical component that transmits only certain wavelengths of light, while reflecting others. In the preferred embodiment, the beam splitter 404 is dichromatic, and transmits all light wavelengths longer than the visible range, along with a large percentage (e.g., 70%) of visible light, while reflecting all other light. Other embodiments may include allow different percentages of the light to transmit.

The mirror 405 can, for example, be an optical component that reflects all light, or all visible light. For example, in order to maximize heat dissipation, the mirror 405 can be a “cold” mirror, which reflects visible light while transmitting heat-inducing NIR and IR light. The heat-inducing NIR and IR light would be passed into the MWIOS 500, where the NIR and IR light can be properly dissipated. Alternatively, the mirror 405 can be a “hot” mirror, which achieves the opposite effect, depending on the orientation of the other components of the embodiment.

In another embodiment, the BS/MC 400 could consist of a single component, the reflective and transmissive properties of which can be changed electronically. For example, a liquid crystal tunable filter (LCTF) device, available from Cambridge Research, Inc, allows the “window” of transmission wavelengths to shift along the spectrum by incorporating a liquid crystal element into a Lyot filter. Products that use this technology are optimized for both the visible and the near-infrared ranges. At the current time, their speed of operation is prohibitively slow for use in an embodiment of the invention; in the future, however, it is expected that this performance time will improve. Another example of such a device is an acousto-optic tunable filter, which contains a piezoelectric transducer bonded to a crystal of tellurium dioxide or quartz and which alters the refractive index of the crystal based on a radio-wavelength input; this, in turn, determines the amplitude and wavelength of light waves passing through the crystal.

Such an electronically-addressable, single-component BS/MC embodiment would function in the same way as described above, either separating the optical path into two separated optical paths, or directing all incoming light so that the MWIOS is avoided.

6.1.5 Multiple Wavelength-Imaging Optical Subsystem (MWIOS)

The optical scope 100a may include a multiple wavelength-imaging optical subsystem (MWIOS) 500 (see, FIG. 1) to acquire simultaneous, high-resolution imagery of the target substrate 101 at the selected wavelengths of interest. (As mentioned above in Section 6.1.2, the conventional optical scope 300 obtains a visible color image of the substrate 101 to be inspected through its imaging device 305.) Light enters the MWIOS 500 when the BS/MC 400 is positioned so that the dichromatic beam-splitter precedes the MWIOS 500 in the optical path.

The MWIOS 500 may be integral with optical scope 100a components (e.g., within the same case, as shown in FIG. 1) or may be housed in a separate case that is incorporated onto the optical scope 100a. In either situation, a seamless optical path is preferably conserved.

The electronic images obtained by the MWIOS 500 can be directed to the system components 100b of the optical scope system 100, including a computational system 700 for analysis and preferably including a user interface 110.

Referring now to FIG. 5, the MWIOS 500 of the invention generally includes the following components:
Entry Lens Set 501
Filter Series 502
Lens Sets 503
Imaging Devices 504
Absorbing Plate 505
Computational System for Data Analysis 700 (see also, FIG. 7)

Entry Lens Set. The MWIOS 500 may include an entry lens set 501. This lens set 501 functions to gather, focus, change the scale of and/or direct the incoming image from the optical scope 100a into the MWIOS 500. This light is suitably directed through the system as an infinity-focused, collimated beam, and thus may require re-focusing to the appropriate distance. The focused light from this lens set 501 can be directed to the first filter 502a in the filter series 502. The lens set 501 may include one or more lenses, as necessary, to introduce the light into the system at a suitable focal distance and/or to improve image quality.

Filter Series. The MWIOS 500 includes a series of filters 502, which serves to extract light of pre-selected bandwidth wavelengths from the incoming image and guide the light to the appropriate imaging device 504. The wavelengths or wavelength bands of the filters 502 are chosen based on the wavelengths of interest for the particular embodiment of the invention, and may, for example, belong to any or all of the ultra-violet, visible, near-infrared or infrared ranges. The wavelengths of interest may be chosen as individual wavelengths, a combination of individual wavelengths, a wavelength band, and/or a combination of wavelength bands.

The filter series 502 may be composed of any optical component that extracts light of a certain wavelength or wavelength band (with the desired bandwidth), such as an interference filter. Other types of filters include dichroic, band-pass and multiple-wavelength filters. The filter series 502 may include as many filters as necessary to accommodate the number of wavelengths or wavelength bands of interest. Alternatively, one or more of the filters may be replaced with an electronically-addressable variable filter, such as the liquid crystal tunable filter or the acouso-optic tunable filter, both of which are described above in Section 6.1.4.

Lens Sets. The MWIOS 500 also includes a lens set 503 that corresponds the filter series 502 and imaging devices 504 in the series. Because focus changes slightly at different wavelengths, the invention may include a focus compensation lens or lens set 503 on some or all of the filter module/imaging device combinations to maintain high-quality, focused imaging. The number and size of these lenses depends on the wavelength and amount of incoming light, the distances from the lens to the filter and to the focal point of the imaging device, and other known optics parameters.

Imaging Devices. In one embodiment, the invention includes a set of imaging devices 504 (preferably monochrome cameras), including one corresponding to each filter 502 and lens set 503 mentioned above. Each lens 503 focuses and directs light of the wavelength or wavelength band extracted by the filter to an imaging device 504. Each imaging device 504 functions to obtain a high-resolution image the light of the wavelength or wavelength band of interest extracted by the corresponding filter 502 from the ultra-violet, visible, near-infrared and infrared ranges. (As mentioned above, the range of resolutions to be employed in embodiments of the invention is expected to range from 4,000 pixels to 16,000,000 pixels.) It should be noted that, given the configuration of the filter series 502 and the imaging devices 504 and 305, it is possible with this invention simultaneously to image the substrate at all wavelengths of interest (with the MWIOS' imaging devices 504), and with wide-band visible light (with the optical scope's imaging device 305).

The specific embodiment shown in FIG. 5 includes five filters, 502a, 502b, 502c, 502d, 502e. Light entering the MWIOS 500 is directed by the entry lens set 501 to the first interference filter 502a, which is oriented to direct any light that passes through the filter 502a also to pass through the lens 503a into the corresponding imaging device 504a. The first interference filter 502a is oriented so that light reflected off of its face is directed to the second filter 502b, which in turn allows only light of the second wavelength band of interest to transmit. Light transmitted through the second filter 502b is directed to the corresponding lens 503b and imaging device 504b, and the remaining light reflected off of the second filter 502b is directed to the third filter 502c. Each filter selects for a different wavelength or band of light. The process can be repeated as many times as there are filters in the series. In various embodiments, the filter series 502 may include 1, 2, 3, 4, 5, 6, 7, 8, 9, 10 or more filters.

Though monochrome area cameras are the preferred embodiment for the imaging devices 504 of this component 700 of the invention, any full-frame imaging device that outputs an electronic image may also suffice to capture image data from the filters and transmit the data to the computational system 700. (By “full-frame”, it is implied that the resolution of the imaging device must be, for example, over about 19,000 pixels, more particularly over about 60,000 pixels. In other embodiments of the invention, the minimum resolution for the imaging devices may be about 100,000 pixels, about 500,000 pixels, or about 1,000,000 pixels, or more.) Moreover, in some embodiments, a single imaging device 504 may be used to capture light from more than one filter/lens combination. In a preferred embodiment, the imaging devices 504 are capable of imaging light from all wavelengths of interest, including for example, the UV, visible, NIR and IR bands.

There are a vast variety of available visible light-imaging cameras suitable for use with the present invention. For instance, other such suitable imaging devices 504 include scan-line cameras, focal plane arrays, and the like. Optimal candidates are digital, have a compact form factor in order to minimize the volume of the optical subsystem, and have an IEEE 1394 “Firewire” interface. For example, a suitable camera that can image both the visible and NIR ranges is a CCD-based camera, which can be optimized to image wavelengths up to roughly 1000 nm.

Imaging devices for imaging the UV, upper-NIR and IR ranges include, for example, InGaAs or cooled-CCD detectors. Preferred devices image the proposed range in a manner that is analogous to the quality of CCD cameras with the visible range. Examples of such include the extended-range Hamamatsu C5840 unit, or Spiricon's 1550 nm Telecom camera, which can image wavelengths from about 1460 to about 1625 nm. Sensors Unlimited offers several InGaAs-based camera models, which range in size and price and can superiorly image the spectrum from 400 nm to 1700 nm, or sub-ranges thereof. Other candidates for imaging the NIR range include those cameras fitted with image-intensifier tubes, as are often used in night vision devices.

Other imaging devices capable of acting as this component of the invention include CMOS-based cameras, line scan cameras and focal plane arrays. Another possible candidate is a mechanically-scanning mirror directed to a detector that receives sequentially-scanned pixels to form a two-dimensional image. Undesirable devices include point-source detectors and other such devices that do not offer a sufficiently high-resolution image.

The electronic imagery from the imaging devices 504 can be transmitted, for example, to the computational system 700 or other system components 100b of the invention for processing.

In particular, the imaging devices 504 should be digitally connected to and provide output to the computational system 700 of the invention.

Flat-Black Absorbing Plate. In some embodiments, the system may include a flat-black absorbing plate 505 at the end of its optical path. The plate 505 functions to collect the unused portion of reflected light, and to prevent the unused portion from straying around the MWIOS encasement, which could corrupt the wavelength-specific images being acquired by the imaging device 504. This absorbing plate 505 may be of any black, light-absorbing material.

6.1.6 Computational System for Data Analysis

The optical scope system 100 of the invention suitably includes a computational system 700, for accomplishing the image processing and analytical functions of the invention. The computational system 700 of the invention accepts input from the optical scope 100a via the MWIOS 500. The computational system 700 of the invention may include one or more computer processors, memory devices, data storage devices, data transmission devices, and output devices, such as the display unit of the user console, as well as printers, and the like. In particular, the computational system will accept data from the imaging devices 504 of the MWIOS 500 (e.g., as described above in Section 6.1.5). Output will include, among other things, the diagnostic imagery as described below in Section 6.1.9. The computational system 700 is programmed to analyze and to compare images of specific light wavelengths.

In a preferred aspect of the invention, these image comparisons are used to reveal the spectral variations of various tissue abnormalities. Information, including bandwidths, polarization and spectral signatures, can be used according to the invention to differentiate normal tissue from abnormal tissue. In one embodiment, the system employs diffuse reflectance spectroscopy (DRS), which provides spectral information that can be linked to the physical composition of the tissue being scanned.

The computational system 700 can measure and compare various spectral parameters, such as scattering and absorption characteristics, across a spectral range. This range may, for example, include wavelengths from the ultra-violet, visible, near-infrared and infrared bands. The MWIOS 500 suitably may include a filter in the filter set 502 for each wavelength being analyzed.

For instance, the wavelength or wavelengths analyzed may be selected based on their facility for imaging the attributes of tissue lesions that differentiate the tissue lesions from healthy tissue. For example, lesions may display excessive angiogenesis, and may consequently contain significantly more hemoglobin than healthy tissue. Excessive angiogenesis may be assessed at wavelengths around the Soret band (420 nm), a very strong absorption band for the heme b protein that primarily constitutes hemoglobin. Other useful wavelengths include the near-infrared [HORN99, ALI04] and infrared [CHIR98] ranges.

In one embodiment of the system, wideband white light of all wavelengths is emitted onto the substrate 101 to be analyzed (the cervix, for example). The returned light is then passed through the MWIOS 500, which may include a series of narrowband interference filters, lenses and cameras that image the light independently at only the individual wavelengths (or wavelength bands) of interest. The system of the invention may then analyze the “spectral signatures” of these images.

The computational system 700 may take the form of any computational unit that is capable of performing sufficient computations to achieve the operations described herein. In a preferred embodiment, the computational system 700 will take the form of a computer, such as palm, laptop or desktop computer. In other embodiments, the computational system 700 may include custom-fabricated integrated circuits or other such devices. In still other embodiments, the computational system 700 may be integrated into a unitary scope device.

Referring now to FIG. 7, the computational system 700 of the invention generally includes the following hardware constituents:
Video Capture Computer Card(s) (not shown)
Processing Unit(s) 702
Software 703
Output Device(s) 704
Input Device(s) 705

Video Capture Cards. The computational system 700 of the invention suitably may include one or more video capture cards or any other device that accepts imagery that has been transmitted from an imaging device and converts the transmitted imagery to data on a computer or other computation unit. The video capture cards are well known in the art, and hence, not shown. The cards function to accept the imagery from the imaging devices 504 of the MWIOS 500 and transmit image data to the processing unit(s) 702 of the computational system 700. The video capture cards may accept and/or transmit data through a cable and/or through wireless means.

Computer processor(s). The computational system 700 of the invention includes one or more computer processors 702 for accepting inputs from the optical imaging subsystem or other input devices 705 and/or analyzing data and/or exporting data to the user console and/or other user interfaces. The computer processor 702 can accept the imagery from the MWIOS via the video capture cards, transfer imagery to storage, process the image data, and/or transfer imagery or other information, such as diagnostic results, to an output device 704. For example, the processor 702 may direct image information to the overlain-eyepiece display 106 and/or to the console display 110.

Software. The computational system 700 of the invention may include software 703 stored on a storage medium or loaded on a computer processor(s) 702 for controlling various operational, computational and/or analytical steps described herein. Software 703 of the invention may be loaded on the computer processor 702 to control operational and/or analytical aspects of the system 700. It will be appreciated that a storage medium, such as an electronic or optical storage unit, including such software 703 suitably also can be an aspect of the invention.

The software components 703 of the invention may be programmed to affect any of the operations described herein. For example, the computational system 700 may include software 703 programmed to achieve any one or more of the following functions:
Image Digitization
Geometric Registration (performed once per optical path configuration)
Intensity Normalization
Examination for Areas of Abnormal Spectral Behavior
Classification of Areas Exhibiting Abnormal Spectral Behavior
Demarcation of Suspected Areas in Visible-Light Color Image
Exporting Method to Displays

Image Digitization. The computational system 700 of the invention may include software 703 for digitizing the images captured by the imaging devices 504 of the MWIOS 500. Digitization will be useful, for example, when analog imaging devices are used rather than digital imaging devices. Digitization can be accomplished using any of a variety of standard analog-to-digital conversion methods. Digitization software can be used to output a digitized data set of each image for use by the geometric registration software.

Geometric Registration (performed once per optical path configuration). The computational system 700 of the invention may include software programmed for geometric registration of the images. Geometric registration creates a two-dimensional mapping of each sub-pixel point in the detection area to a pixel in the frames of each of the imaging devices 504. (This task can be simplified by having all imaging devices share an optical path.) This subroutine would function to ensure that all points in space are consistently accounted for in each image. A number of different techniques for registration exist; for example, a pre-determined look-up table may be compiled by imaging specially designed targets (equipped with fiducials) in connection with fiducial detection algorithms.

It should be noted that this step need only be performed once for a given optical path, since the two-dimensional mapping of the cameras will presumably not change. If the optical path is modified in any way from the previous geometric registration's configuration, the step will need to be repeated.

Intensity Normalization. The computational system 700 of the invention may include a method for normalizing the intensities in the images from the imaging devices 504. This step would function to eliminate or minimize variations in: the spectral response of the imaging devices 504; the spectral output of the light source 301; the degree of absorption through the filters 502; the overall expected lighting conditions; tissue pigmentation; and other factors.

Normalization may be achieved through a variety of means. In one such method, a uniform flat target is imaged by each imaging device 504 and filter 502 combination and under varying illumination intensities and exposures. From analysis of this set of flat-field calibration imagery, it is possible to calculate per pixel (or per group of pixels to average out noise) normalization coefficients for each imaging device 504. This method of intensity normalization includes a subroutine to remove areas of the monochrome images that are either above or below set boundary levels of intensity. This is done so that areas of the image which receive levels of light too high or too low for proper image processing are removed before the computation across the image is done. Such areas could be highlighted in a different way from the manner in which suspected abnormalities are highlighted (as described below), distinguishing them for the user. If the user so chose, he/she could position the tissue in such a way as to provide more or less light to the unprocessed areas.

Intensity normalization could be carried out automatically using the embedded software architecture of the invention. Alternatively, the invention could support a mechanism whereby the user selects an area of the image which he/she knows to be healthy, and the optical properties of this area would be used to normalize the remaining sectors of the image.

Examination for Areas of Abnormal Spectral Behavior. The computational system 700 of the invention includes a method for recognizing spectral anomalies across the narrowband wavelength-images of the substrate 101, for instance tissue, under inspection.

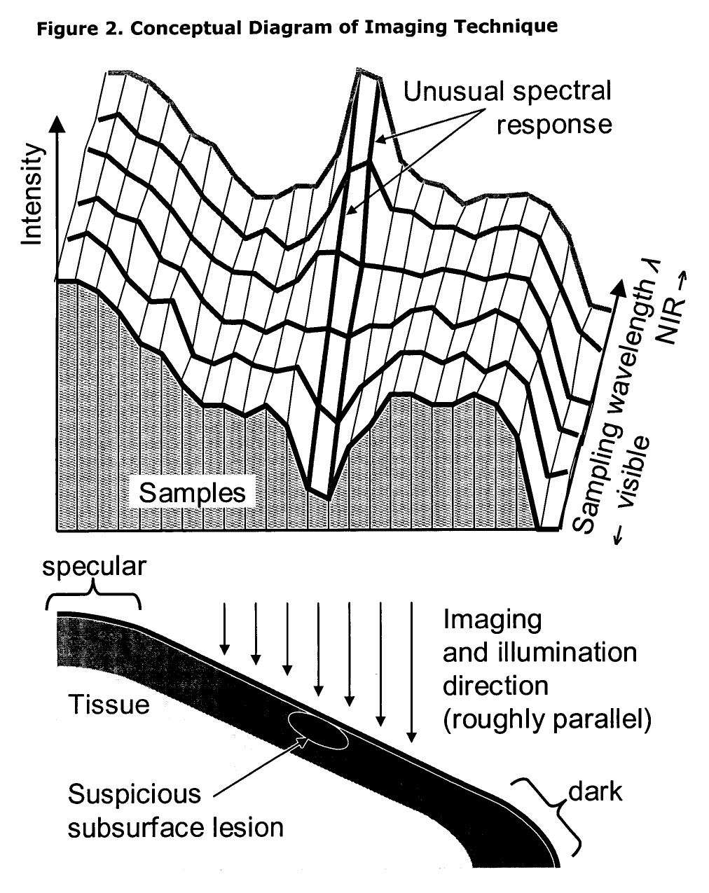

Referring now to FIG. 2, the graph shows an example of the data collected by the invention. In the system 100 of the invention that detects cervical lesions, for example, the “samples” axis represents an imaginary “line” of tissue substrate 101 that the system 100 is scanning. (In actuality, the system 100 will scan many such lines simultaneously—as many as the vertical resolution of the imaging devices in the MWIOS 500.)

The spectral signature of each “point” or pixel in the tissue sample “line” can be read by looking at the corresponding 2D graph (“sampling wavelength” axis versus “intensity” axis) at that point. The “sampling wavelength” axis will have exactly as many points as there are wavelengths of interest in the embodiment of the invention. (In a currently preferred embodiment for cervical cancer detection, this number is five: 420 nm; 500 nm; 849 nm; 956 nm; and 1450 nm.) Therefore, at a “samples” value of 15 (pixels) and a “sampling wavelength” value of 1450 nm, the “intensity” plot then communicates the intensity level of the “point” on the tissue sample “line” at 15 pixels from the origin in the image taken at the 1450 nm wavelength.

The depiction of a sample tissue (here, a cervix) directly below the “samples” axis is meant to indicate a point-to-point correspondence of the tissue location to the spectral signature shown in the graph. (This indicates that the imaging plane is roughly parallel to the tissue surface.)

It is noted that in the “tissue” representation at the bottom of the diagram that the tissue sample becomes more parallel to the imaging “samples” axis at the left end of the representation. (This is in line with the cervix becoming parallel to the entry lens at its center.) Light at this location would reflect directly back into the entry lens, causing specular highlights that would prevent the area from being imaged; this is reflected in the “intensity” axis of the plot, where the intensity values across the wavelength spectrum are at maximum value.

Likewise, the right end of the representation corresponds to the outer walls of the cervix, which are perpendicular to the imaging plane. It is noted that in this example, the intensity values in the plot for this location indicate that the images are receiving too little light to image that area properly.

By analyzing these spectral signatures across the entire area of the substrate, the system 100 is able to distinguish characteristics thereof for the user. Continuing with the invention's cervical cancer detection embodiment, the system 100 will be able to determine whether any sub-areas of the cervix contain pre-cancerous or cancerous lesions. In order to equip the system 100 with such a capability, it is desirable to determine through ratiometric (or “principal component”) analysis a weighted combination of coefficients for the spectral signature's wavelengths that will accommodate a broad range of patients. (Pre-determined ratiometric analysis is described in more detail in Section 6.2.1 below.)

It is noted that the amount of area of substrate 101 to be examined by the scope 100a can vary by application. For example, in the embodiment of the invention wherein the scope 100a observes the epidermis, the area to be examined may range from 50 mm to 80 mm at its widest cross-section, while this area for the embodiment of the invention wherein the cervix is being examined may range from 15 mm to 30 mm. In still another embodiment of the invention, wherein a narrow range of the thoracic or gastrointestinal cavity is being examined, the area may be limited to 2 to 15 mm at its widest cross-section.

Classification of Areas Exhibiting Abnormal Spectral Behavior. The computational system 700 of the invention may include a method to classify areas of abnormal spectral behavior. This classification method would serve to distinguish between suspected abnormalities in terms of, for example, condition, stage of development or severity. This would be accomplished by analyzing the conformance of the suspected area's spectral signature to that for each stage or condition.

Demarcation of Suspected Areas in Visible-Light Color Image. The computational system 700 of the invention suitably may include a method to demarcate the pixels of the final diagnostic image that contains suspected abnormalities from the rest of the organ under inspection, in order to convey the diagnostic information to the user of the system 100. This can be achieved, for example, through assigning the pixels a distinguishing color; the color may be assigned based on the suspected abnormality's classification, as determined by the previous section's subroutine.

Data Exportation to Displays. The computational system 700 of the invention suitably may include may include output devices 704, such as a display device, to provide a method to export diagnostic data to the user. This method would function to allow the user to view the analysis of the computational system.

6.1.7 Distance Indexing Mechanism (Optional)

The invention can optionally include a distance indexing mechanism. Such a component would function to track the distance from one location pertinent to the invention's function to another location. For example, in the embodiment of the invention that is used to detect colorectal lesions, the distance might be from the body's exterior to a probe containing the entry lens, which would detect a suspected lesion along the path of the colon.

This component may be actuated in a number of different ways. For example, the probe containing the entry lens may be marked along its side, so that distance could be read manually at places where a lesion is suspected. Alternatively, the system could contain an electronic method for measuring distance traveled, such as a tracker or other such device.

6.1.8 Overlain-Eyepiece User Interface

The optical scope system 100 of the invention may include a physical user interface to allow the user to view the data through an eyepiece 107, 306, which is the standard interface on many optical scopes (e.g. a colposcope for cervical lesion detection). In some embodiments, the interface is an overlain-eyepiece display 106 (e.g., as illustrated in FIG. 1). The overlain-eyepiece display 106 may include a computer-driven image display device, and may also include a servo motor or other actuator.

Computer-Driven Image Display Device. The system 100 of the invention may include a computer-driven imaging subsystem, e.g., in embodiments that include an overlain-eyepiece display 106. The imaging subsystem can be used to present imagery from the computation system 700 to the eyepiece 107, 306 of the optical scope 100a.

Dial 401 (see, Section 6.1.4) can be included to permit the user to select the mode of operation in which digital imaging is active. The computational system 700 can affect the motor to position the image display device in the optical path leading to the eyepiece 107, 306, thereby blocking the light coming from the scope optics. Alternatively, the position of the image display device can be effected by mechanical means. Dial 401 (see Section 6.1.4) can also be used to permit the user to inactivate digital imaging.

In some embodiments, the back of the image display device 110 can be painted or otherwise coated with a light-absorbing coating, such as a flat-black coating, to absorb the light being directed to it by the beam splitter 304, and thereby to prevent corruption of the imaging device 305 image caused light from reflecting back to the beam splitter 304 and into the field of view of the imaging device 305.

The imaging device may be, for example, an LCD device, a ferro-reflective display device, or another image-producing display device.

Motor or Other Actuator. The embodiment of the invention that contains this overlain-eyepiece display 106 can include a motor, such as a servo motor, or other such actuator. The motor or other actuator will move the image display device in and out of the optical path. In one embodiment, the motor will receive its control signals from the computational system 700, based on the position of the dial 401. Alternatively, the computational system 700 will be programmed to permit the user to select the mode of operation (i.e., move the image display device in or out of the optical path) using an input, such as an input from a mouse, keyboard and/or other input device, e.g., by “clicking on” or selecting a virtual online switch displayed on a display device.

6.1.9 Software-Driven Console User Interface

The invention may also include a user interface console 110. The user interface console 110 may be software driven, e.g., via software loaded in a processor and driven by various input devices, such as a touch-screen input device, a mouse, joystick, or various switches or dials, that enable the user to provide inputs to the system. The input devices may be coupled to the processor by various means known in the art, and may be wireless. In some embodiments, input devices will be mounted on the optical scope 100a.

The user inputs may, for example, instruct the system to control various system capabilities, such as operational capabilities and/or analytical capabilities. For example, such input means may enable the user to select various modes of operation, change display options, move the image display device in and out of the optical path, move the dichromatic beam splitter 404 and/or the mirror 405 in or out of the optical path, and the like.

The user interface console 110 may allow the user to select between various modes of operation. For example, the user console may permit the user to select continuous processing mode, in which current imagery is acquired by the MWIOS 500, analyzed in the computational system 700, and displayed on the user console 110. Alternatively, the user console may permit the user to select a single-frame processing mode, in which the process is performed only when triggered through software by the user.

The user interface console 110 may include one or more output devices for communicating information to the user or others, such as display devices, printer devices, as well as devices for transmitting data to other computers (e.g., via the Internet), such as modems.

The interface may permit the user interactively to display, to manipulate and/or to analyze the images. For example, the user may use various input devices associated with the user console 110 to display an interactive spectral response utility showing the spectral behavior of the acquired samples along a user-positioned line segment or other shape outline in the image, e.g., as illustrated in FIG. 2.

Preferably, the display will permit the user to show the intensity-normalized brightness values (axis marked “Intensity”) at all wavelengths of interest (axis marked “Sampling Wavelength 8”) along the samples, or pixels (axis marked “Samples”) intersected by the line segment. By moving and/or rotating the line segment with an input device (e.g., a simple mouse-based interface), the user can visualize the spectral response along arbitrary curves on the surface of the tissue (a line in the raw optical scope image projects to a curve on the surface of the tissue, as shown in FIG. 2). By changing the length of the line segment, the user will be able effectively to zoom in on an area of interest and closely inspect the spectral response of suspicious locations on the tissue (e.g., on the cervix).

Computational system 700 may also be programmed to permit the user to use console 110 to explore raw individual-wavelength images, to magnify images, to record and to store data, and the like.

6.2 Substrate Analysis

The invention provides methods for using the apparatus 100a and system 100 of the invention to analyze optically a substrate 101. The methods of the invention generally involve emitting light upon a substrate 101, collecting light reflected from the substrate 101, and recording and analyzing the light to provide information about the optical characteristics of the substrate 101.

The ensuing examples focus on the embodiment in which the substrate 101 is a tissue substrate (human tissue or animal tissue), and the analysis relates to optical characteristics useful for identifying tissue abnormalities, such as pre-cancerous and cancerous lesions, glucose abnormalities and burn injuries. However, it will be appreciated that the methods of the invention will be useful in the analysis of other substrates 101 as well, including for example, in vitro tissue samples; manufactured materials such as plastics and metals; soil or other materials.

6.2.1 Lesion Detection and Identification

The methods of the invention can be employed in the detection of tissue lesions.

Operation in Principle. The embodiment of the invention that detects tissue lesions operates in principle in the following way: certain wavelengths of light are selected for their known, distinct behaviors when interacting with healthy tissue and with tissue lesions; these wavelengths are extracted from light reflected off of the tissue and imaged; the wavelength-specific images are studied for the pre-determined characteristics associated with tissue lesions.

For example, referring now to FIG. 2, a tissue sample is schematically illustrated at the bottom of the figure, and above the tissue sample, its spectral signature across several chosen wavelengths is presented. In the lowest wavelengths of this sample (i.e., those toward of the end of the “sampling wavelength” axis marked “visible”), the intensities for the samples relating to the lesion are much lower than those for healthy tissue. A demonstrated explanation for this is that the lower wavelengths tend to absorb into hemoglobin, which is present in greater quantities in lesions than in healthy tissue, so less light is reflected. Likewise, these intensities may become higher than the healthy samples as the wavelength increases; it has also been demonstrated that the higher wavelengths tend to scatter more in lesions, so more reflected light of these wavelengths may be received by a detector.

However, it is not expected that such monotonic behavior will be consistently encountered; due to the complexity of light's interaction with the realm of tissue, for instance human tissue, healthy tissue on one patient may exhibit a very different spectral signature from equally healthy tissue on another. Because of this, embodiments of the invention to be used for tissue lesion detection should first undergo extensive ratiometric analysis, as detailed in the following section.

Ratiometric Analysis. The system of the invention can be programmed to distinguish spectral abnormalities associated with the specific lesions being targeted across a broad range of patients. This multi-wavelength response pattern range can be determined through ratiometric (or “principal component”) analysis of spectral data, which will result in a “z-score”—i.e., a weighted linear combination of the intensity values of each wavelength in ratio to each other.

The magnitude of the coefficients is determined by how heavily the ratio of wavelength intensity influences the spectral signature of the given lesion type; each lesion classification to be detected by the system will be assigned its own z-score. For example, in an embodiment of the system wherein the selected wavelengths are 750 nm, 850 nm, and 950 nm, the z-score would take the form
Z=α·R₇₅₀R₈₅₀+β·R₇₅₀R₉₅₀+γ·R₈₅₀R₉₅₀
where the coefficients are represented by the Greek letters α, β, and γ, and where R₇₅₀, R₈₅₀ and R₉₅₀ indicate the normalized intensity responses at the wavelengths 750 nm, 850 nm, and 950 nm, respectively.

This data can be obtained by performing statistical analysis on a large database of spectral signatures taken in vivo from various patients, along with the correspondent pathological results (as determined by, for example, biopsy). Such a database should contain many hundreds, more particularly thousands, of samples, and the demographic array of patients included should be inclusive of different races and ethnicities, in order for the system to perform in a desirably accurate manner.

Lesions can be automatically detected by comparing the data obtained from target tissue areas to these standardized data patterns. Thus, in one aspect of the invention, a potential lesion is detected when a tissue region is identified which has a multi-wavelength response pattern that (a) significantly matches the standardized data pattern of a target lesion, and/or (b) differs significantly from the multi-wavelength response pattern of comparable normal tissue in the subject.

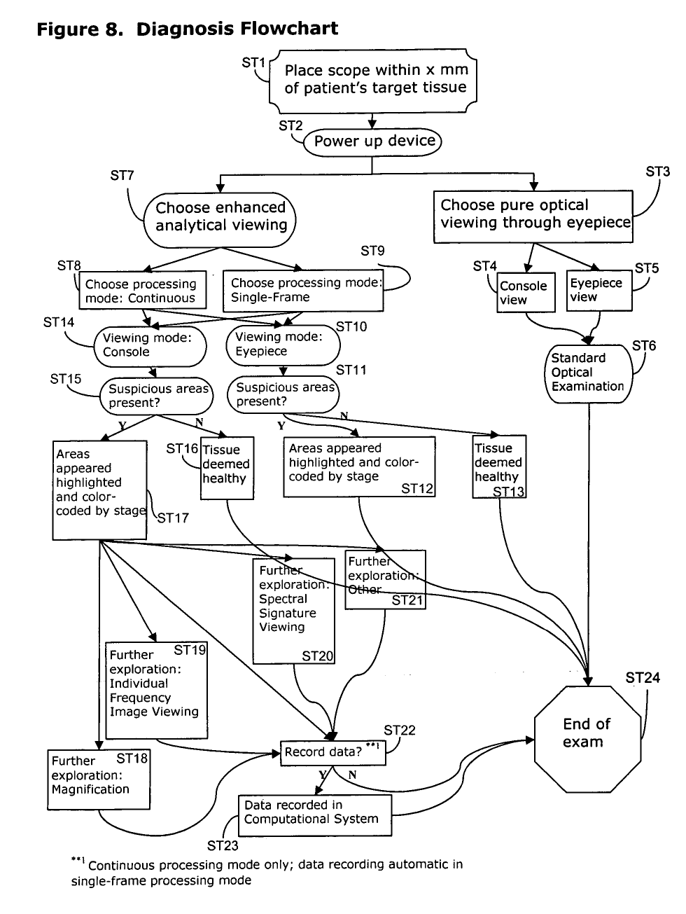

Practical Implementation. FIG. 8 presents in flowchart form a typical procedure for using a system of the invention, such as the system 100 described in FIG. 1, for detecting a tissue lesion.

Referring to FIG. 1 and FIG. 8, according to the method, the entry lens 103 of the optical scope 100a is positioned at an appropriate distance from the target tissue substrate 101 on the subject (ST1, ST2). This positioning step may be preceded by appropriate patient positioning or surgical steps necessary to expose or otherwise provide access to the target tissue substrate 101. Exact positioning will vary depending on a variety of factors, such as the brightness of the light being used and the type of tissue substrate 101 being analyzed. The optical scope 100a is appropriately positioned when the scope can collect sufficient light to perform its function, i.e., gather sufficient reflected light to identify a lesion, and preferably sufficient light information to permit the system 100 to identify the lesion and differentiate the lesion from other lesions.

It will be appreciated that for embodiments of the invention that detect lesions on large organs, such as the colon, this positioning step may need to be repeated several times during the examination. In this case, if the system of the invention includes a distance indexing mechanism as described in Section 6.1.4, then the distance for each imagery set acquired will be noted, through manual and/or automatic means.

Certain areas of the tissue may exhibit specular highlights, so that measurements cannot be taken, while other areas, particularly those that are either shadowed or nearly tangential to the oncoming light, may not reflect sufficient light. To inspect such areas, the user can manipulate the tissue being inspected (the cervix, for example), or alternatively, the light source, so that the tissue is adequately exposed and illuminated to reflect sufficient light to permit effective analysis.

By appropriately positioning the BS/MC 400, the user selects whether to use the optical scope 100a as a conventional scope or to use the enhanced lesion-identification capabilities of optical scope 100a. In the latter case, light encompassing all wavelengths of interest is emitted on the tissue 101. In a preferred embodiment, wideband white light from any or all of the ultraviolet, visible, near-infrared, and infrared ranges is emitted onto the tissue 101. Individual wavelengths will absorb into or reflect off of the tissue to different degrees based on the physical composition of the tissue 101.

The BS/MC 400 of the invention is arranged in the optical path and directs a portion of incoming visible light via the scope optics for viewing via eyepiece 107, 306, and the remainder of the light into the MWIOS 500. In a preferred embodiment, the amount of light directed via the scope optics for viewing via eyepiece 107, 306 is minimized, and the amount of light directed into the MWIOS 500 is maximized to provide maximal diagnostic image resolution in the MWIOS 500.

For example, in one embodiment, the BS/MC 400 directs at most 30% of incoming visible light via the scope optics for viewing via eyepiece 107, 306, and the remaining light (at least 70% visible) into the MWIOS 500. Preferably all or substantially all non-visible light is directed into the MWIOS 500. Thus, as another example, the BS/MC 400 directs at most 30% of incoming visible light via the scope optics for viewing via eyepiece 107, 306, and the remaining light (at least 70% visible and 100% NIR and IR) into the MWIOS 500.

Light of the first individual wavelength or wavelength band travels through the first filter 502a and lens 503a set to the first imaging device 504a, which images the target tissue 101 at only the first wavelength band of interest. The remaining light is directed to the next interference filter 502b, which in the substantially the same manner results in the imaging of the second wavelength of interest. This process is repeated as many times as there are filters 502 or wavelengths of interest. The images collected by the imaging devices 504 are transmitted to the computational system 700 for analysis.

The transmitted narrowband-wavelength images are acquired into the computational system 700 video capture cards, and are digitized if not already in digital form. This image set is then analyzed in the image processing pipeline described above in Section 6.1.6.

In near-real time, a user, such as an administering physician or technician, can view the resulting diagnostics of the invention. Using an optical device such as a “half-silvered mirror” or an image display device, the suspected lesions can be superimposed over the view seen by the optical scope 100a standard eyepiece view, which most users will already be accustomed to using. An interactive console 110 can also be used to display the results, allowing for extended investigation of the diagnostic data. The system 100 can be backward-compatible, or configured to permit the user to disable the analytical capabilities of the invention and instead operate as a conventional optical scope, by using a mechanism to block temporarily the reflected light from entering the MWIOS 500.

For example, the optical scope 100a can be configured to permit the user to view results, for instance, a camera frame with the diagnostic data superimposed on the unmodified camera image of the tissue, through the eyepiece 107, 306 of the optical scope 100a. This option can be achieved, for example, by positioning a digital imaging device and a half-silvered mirror to superimpose the diagnostic imagery onto, and optically combine it with, the optical path of the optical scope 100a.

Imagery from the optical scope 100a may also be displayed on an output display unit 110, such as a CRT or flat panel display of a computer system. This approach provides a convenient way for the user to view the data, and the data may be further processed. The diagnostic results are presented to the display unit by the processor, preferably loaded with a software module of the invention. The software can provide the user with data manipulation capabilities, such as magnification, isolation of individual conditions.

In some embodiments of the invention, the system 100 includes only the capability of viewing the image through the optical scope 100a. In other embodiments, the system 100 includes only the capability of viewing the image through a display unit 110. In still other embodiments, the system 100 includes both the capability of viewing the image through the optical scope 100a and via a display unit 110.

In certain embodiments, after the images have been viewed and/or recorded, the system 100 immediately outputs information characterizing any lesion identified. Such information may, for example, include a map of the tissue analyzed identifying the specific location of any lesions identified. The information may also include diagnostic characterization information, such as information about the type of lesion identified or information about types of lesions ruled out by the analysis.

The system 100 may characterize a lesion as a specific lesion type, or may characterize the lesion as a member of a certain set of lesions which share the characteristics of the lesion in question. The system 100 may further output information showing the statistical probability that a lesion is of a certain type, e.g., “the lesion has the characteristics of a lesion of type A; 80% of lesions having these characteristics are of type A” or “the lesion has the characteristics of a lesion of types A, B and C; 70% of lesions having these characteristics are of type A, 20% are of type B, and 10% are of type C.”

Based on the results of the analysis, the user can directly report the results to the subject. Thus, for example, in one embodiment, the invention provides a diagnostic method in which a system of the invention is used to image tissue of a subject; the image is analyzed to produce a diagnosis; and the diagnosis is communicated to the subject on the same day, preferably almost instantaneously.

In another embodiment, the invention provides a diagnostic method in which a system of the invention is used to image tissue of a subject; the image is analyzed to determine whether a biopsy is needed; and if the analysis indicates that a biopsy is needed, the biopsy procedure is performed on the same day as the analysis.

In still another embodiment, the invention provides a surgical method in which a system of the invention is used to image tissue of a subject during a surgical procedure; the image is analyzed to determine whether, for example, certain tissue should be removed; and if the analysis indicates that tissue should be removed, the tissue is removed during the surgical procedure or in a subsequent surgical procedure.

6.2.2 Direct Characterization of Tissue Using Standard Laboratory Classifications

The methods of the invention allow for more specific characterization of an abnormality once it has been targeted. For example, in the embodiment of the invention to detect cervical cancer (described in the following section), a suspected lesion on the cervix may be classified according to the Bethesda system, which is used by most laboratories that categorize the results of the Papanicolaou test. Alternatively, the results could be classified as they would be in standard white-light colposcopy, of which this embodiment of the invention is an enhancement.

6.2.3 Colposcopy Enhancement for Rapid Cervical Cancer Screening

The methods of the invention include a rapid screening method for lesions on the cervix, such as cancerous and pre-cancerous lesions, based on ca conventional colposcopy exam. The system of the invention can produce a wide-area, high-resolution screening capability using full-frame (i.e. high-resolution) imaging devices. This wide-area screening capability can permit the entire cervix to be imaged in the frame at once. The rapid cervical cancer screening method generally involves the following steps:

Positioning the scope. The user positions the diagnostic colposcope in sufficient proximity to the cervix to permit imaging, and the user powers up the device. Preferably, the diagnostic colposcope is within about 20 mm to about 30 mm of the patient's cervix.

Selecting an operation mode. In some embodiments, the user selects an operation mode, e.g., continuous processing mode or single-frame processing mode.

Analyzing wavelengths. The system can analyze the individual wavelengths of interest for cervical cancers and pre-cancers for abnormalities.

Viewing the results. The user can inspect the results on either the eyepiece 107, 306 or console display 110. If using the console display 110, the user can perform useful manipulations of the imagery with the custom software described above in Section 6.1.9.

Determining next steps. Based on the results, the user may diagnose the condition. If visual data reveal cancerous or pre-cancerous lesions, appropriate steps can be taken in accordance with standard medical practice for the treatment of such lesions. A biopsy of the affected area may be obtained for confirmation of the results. Appropriate surgical procedures may be scheduled.

6.2.4 Colonoscopy Enhancement for Colorectal Cancer Detection

Another embodiment of the invention involves the detection of colorectal pre-cancers and cancers. This embodiment of the invention can be implemented in hardware by means of a flexible endoscope (along with integrated fiber optic illumination) with an MWIOS 500 of the invention. Like the cervix, the colon and rectum are covered in epithelium, and cancerous and pre-cancerous lesions have optical characteristics that are different from those of healthy tissue. Thus, colorectal lesions can be investigated in substantially the same manner as those on the cervix.

6.2.5 Other Analytical Targets and Conditions Diagnosed

While the current specification describes the invention using cervical and colorectal lesions as examples of lesions detectable by the system, it will be appreciated that the system can be adapted to target lesions on any exposed tissue surface, such as any epithelial tissue. The epithelium covers most of the accessible internal organs in the body (e.g. thorax, rectum, colon, cervix, vagina, skin). Furthermore, the invention may also be used to analyze bum injuries and glucose abnormalities. Moreover, it will be appreciated that not all of the steps described are required. Also, the steps described may be accomplished in various orders to obtain substantially the same results, and/or some steps described may be accomplished in parallel.

Preferred analytical targets for the invention include all organs lined in epithelial tissue, including, but not limited to, endothelial tissue; simple or stratified epithelium; squamous, cuboid or columnar epithelium; and ciliated or glandular epithelium.

Examples of specific target organs include organs of the thoracic cavity, other organs in the gastrointestinal tract (e.g. anus, rectum, etc), and the epidermis. Examples of cancers that can be diagnosed according to the methods of the invention include, for example, carcinomas, such as adrenocortical carcinoma, which arises from the adrenal cortex; thyroid carcinoma, which arises from the thyroid; nasopharyngeal carcinoma, which affects the nose and pharynx; malignant melanoma, a cancer of the skin; skin carcinomas, such as basal cell carcinomas; and other carcinomas.

Another possibility for use of the invention is in the surgical removal of the lesions or tumors being screened. Such a device also should be very useful for ensuring that the entire transformation area of the pre-cancer or cancer is extracted.

### 7 EXAMPLES

7.1 Optical Scope and System for Use in Detecting Cervical Pre-cancers and Cancers

In one embodiment, the system of the invention is constructed as follows:

7.1.1 Embodiment 1

In this embodiment, the system is designed to detect cervical pre-cancers and cancers. Several wavelengths of interest are known in the art and thus have already been identified for inclusion into such a system: 420 nm [GEOR02, MIRA02], 500 nm [NORD01], 849 nm [HORN99], 956 nm [HORN99], and 1450 nm [ALI04].

The MWIOS 500 can be constructed with five interference filters 502 for isolating and imaging the wavelengths of interest. The wavelengths of interest can be transmitted to an image processing pipeline, which will determine the optical properties of the target substrate. The system can be calibrated using tissue phantoms (as explained below in Section 7.2), and on in vitro tissue samples (as explained below in Section 7.3).

Two optical paths for the MWIOS are now described.

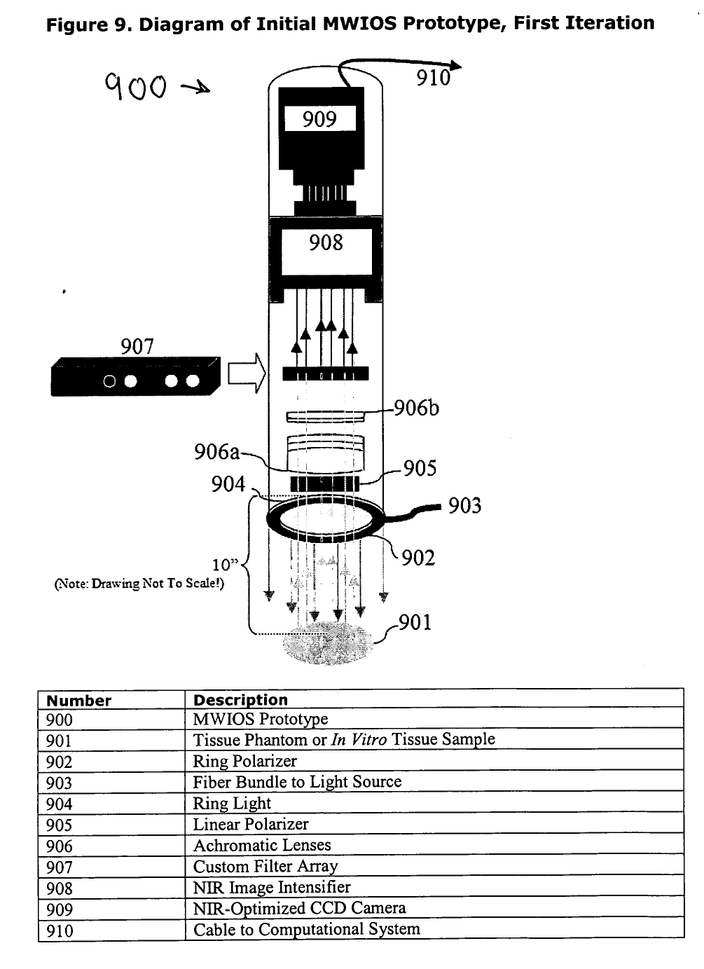

Referring now to FIG. 9, this embodiment depicts an MWIOS prototype 900, which generally includes the following components for analysis of a tissue phantom or in vitro tissue sample 901:
Tissue Phantom or in vitro Tissue Sample 901 to be analyzed
Ring Polarizer 902
Fiber Bundle to Light Source 903
Ring Light 904
Linear Polarizer 905
Lens 906 (preferably achromatic)
Filter Array 907 (preferably custom)
Image Intensifier 908 (preferably NIR)
Camera 909 (preferably CCD camera that is NIR-Optimized)
Cable to Computational System 910

More particularly, the MWIOS prototype 900 employs an inexpensive image intensifier 908 and an NIR-optimized CCD camera 909, which is able to image both visible and (at a lower quality) near-infrared wavelengths, in order to obtain preliminary performance feedback.

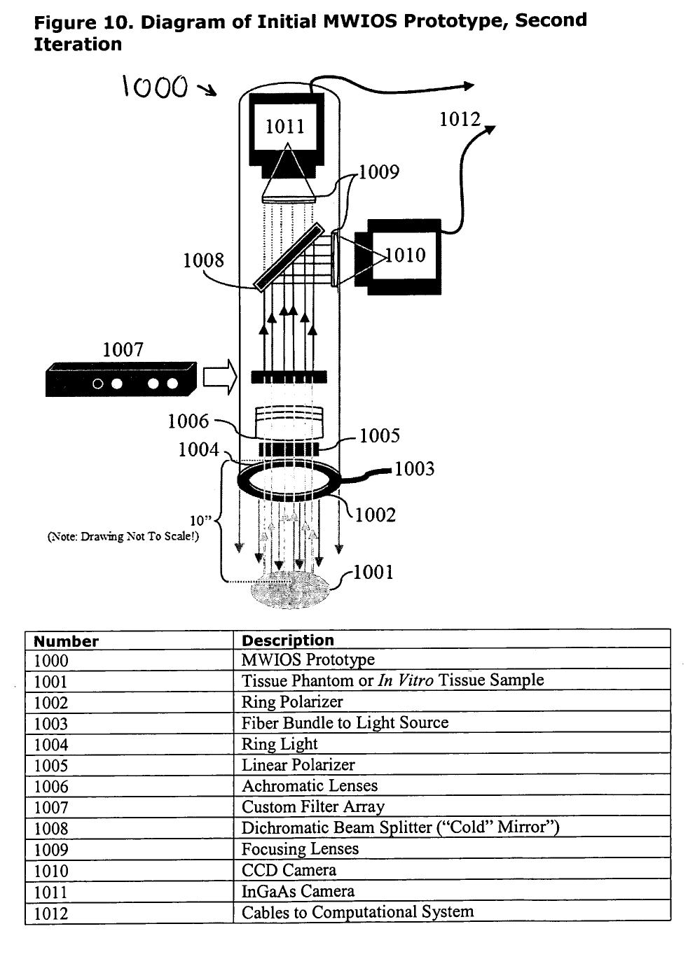

Referring now to FIG. 10, instead of the MWIOS prototype 900 depicted in FIG. 9, shown is a (more costly) MWIOS prototype 1000, which generally includes the following components for analysis of a tissue phantom or in vitro tissue sample 1001:
Tissue Phantom or in vitro Tissue Sample 1001 to be analyzed
Ring Polarizer 1002
Fiber Bundle to Light Source 1003
Ring Light 1004
Linear Polarizer 1005
Lens 1006 (preferably achromatic)
Filter Array 1007 (preferably custom)
Beam Splitter 1008 (preferably dichromatic and preferably “cold mirror”)
Focusing Lenses 1009
Camera 1010 (preferably CCD camera)
Camera 1011 (preferably InGaAs camera)
Cable to Computational System 1012

More particularly, the MWIOS prototype 1000 generally employs an InGaAs-based camera 1011, which is a sophisticated new technology for superior imaging of wavelengths up to 1800 nm. However, the InGaAs camera 1011 is not sufficiently sensitive to the lower visible wavelengths (420 nm and 500 nm), so the MWIOS prototype 1000 also includes a CCD camera 1010 and a directive dichromatic beam splitter 1008 (or “cold” mirror, as it reflects visible wavelengths and transmits the NIR and IR ranges), as shown in FIG. 10.

Other than this difference for the MWIOS prototype 900 and the MWIOS prototype 1000, the two optical paths can be identical.

Similar to what is described above in Section 6.1.1, the following discussion applies respectively to the MWIOS prototype 900 of FIG. 9 and the MWIOS prototype 1000 of FIG. 10.

A standard ring light source 904 or 1004 is passed through a polarizer 902 or 1002 and illuminates the target tissue 901 or 1001. The reflected light passes through a linear polarizer 905 or 1005 and one or more achromatic lenses 906 or 1006, which focus the wide band of wavelengths passing through the lens. The focused light then passes through one of the interference filters in the custom filter array 907 or 1007, which can contain the wavelengths of interest. This array 907 or 1007 typically is altered manually and the target re-imaged in order to obtain images at all wavelengths of interest.

Once a full set of imagery is acquired, the computational system can analyze the set of imagery for optical characteristics. The images are registered and intensities normalized; the images are then be analyzed for areas whose spectral characteristics closely match those determined to be associated with abnormalities in initial calibration experiments.

These areas are highlighted and presented to the user on a console screen.

7.1.2 Embodiment 2

This embodiment is based on the schematic presented in FIG. 1. A standard colposcope, based on well-established colposcopic optical diagrams, can be constructed, into which all ancillary components associated with the invention can be integrated. The entrance portion of the optical path will be similar to the MWIOS described in Section 7.1.1, including a light source 102, a tissue target 101, an entry lens 103, 302 and an achromatic lens 104 for wide-band distortion correction. In this embodiment, in order to accommodate backward compatibility to a conventional optical scope, the full-system's optical path must include a BS/MC 105 after the entry lens set 103, 302.

The MWIOS 500 of this embodiment may include of four high-quality NIR-optimized CCD cameras, to individually image the 420-nm, 500-nm, 849-nm and 956-nm wavelengths, and one InGaAs camera, to image the 1450-nm wavelength. While the images at 849 nm and 956 nm would perhaps be better served by InGaAs cameras, practical budgetary limitations must be taken into account at this early stage. Additional InGaAs cameras may be employed.

The computational system 700 of this embodiment will operate in substantially the same manner as that of the Embodiment 1 described above. The image set obtained will be registered and intensities will be normalized. The images will then be analyzed for areas whose spectral characteristics closely match those determined to be associated with abnormalities in initial calibration experiments; any such areas will be highlighted for presentation on the user interface.

Custom housing can accommodate all physical components of the optical scope 100a. In this embodiment, the system components 100b are provided separately. The unit will be mounted on a lockable rolling stand 108, for ease of use in clinical environments.

Various embodiments of the invention can undergo testing to maximize their design effectiveness.

7.2 Tissue Phantom Tests

Tissue phantoms with an array of scattering and absorption properties can be developed for both calibration and testing of all instruments described in above. The phantoms act as inexpensive optical proxies for real tissue by presenting comparable absorption and scattering properties at each of the chosen wavelengths.

The components of the phantoms can be chosen based on their own absorption and scattering coefficients; they can include one or more of: Intralipid™, a fat emulsion that mimics bulk tissue; polystyrene spheres, which serve to scatter light in a manner predicted by Mie theory; and hemoglobin, the component of blood that absorbs light in narrow bands; and India ink, which absorbs light over a broad spectral range.

All embodiments of the invention should first be calibrated to ensure proper performance. To simulate accurately the coefficients of the health conditions to be detected with the imaging instrument, it will be useful to alter systematically the scattering (μₛ′) and absorption (μₐ′) coefficients across the range expected in the prepared phantoms, and to record correspondent changes in the intensity values of the system's imaging device.

For example, Hornung [HORN99] reports that normal cervical tissue will exhibit a reduced scattering coefficient μₛ=0.498 mm⁻¹ and an absorption coefficient of μₐ′=0.057 mm⁻¹ at 956 nm while abnormal tissue will show decreases in these values by up to 30%. Based on the model of Van Staveren [VANS91], the stock 10% Intralipid™ will possess a reduced scattering coefficient of approximately μₛ′=10 mm⁻¹ at 956 nm and thus will be diluted 20:1 to simulate normal tissue scattering. The solution will be further diluted to simulate abnormal tissues.

The absorption characteristics can be varied by adding small amounts of India Ink to the phantoms. India Ink in concentrations of 1 mL/L will produce the absorption coefficients needed to simulate tissues at the chosen wavelengths. The phantoms can be prepared to simulate the range of expected scattering and absorption properties in 5% intervals relative to the average measurements listed by Hornung [HORN99] up to a 50% variation. As previously stated, the system will observe and record each of these combinations; the spectral variation of the absorbers (measured as unusual combinations of intensity ratios) will enable the system to distinguish between increased absorption and increased scattering.

Another battery of tests can be carried out on imagery that simulates the more complex appearance of in vivo tissue, where most of the imaged area consists of healthy tissue and only sporadic fragments of diseased tissue may be encountered. Using the stored images of Petri dish phantoms differing only in composition, it is possible digitally to create images of phantoms conforming to the above description. This will be accomplished by digitally compositing over an image of the “ealthy” phantom a fragment of a pre-cancerous lesion phantom image acquired under the same lighting conditions and using the same filter. (Gradients of intermediate- stage lesion and/or advanced-stage lesion imagery can also be used, perhaps most accurately mimicking the physical transformation zone of a cancerous lesion.) By doing this for all inspected wavelengths, the user can build sets of synthetic test data for the system. Furthermore, to simulate brightness variations, the user should also acquire imagery of each of the phantoms using a range of illumination intensities. By digitally blending portions of the resulting darker and brighter images, the user can create test imagery that simulates intensity gradients of curved surfaces, without having physically to alter the phantoms or re-acquire any imagery.

7.3 In Vitro Tests

Various embodiments of the invention can be optimized using in vitro tissue samples. Such samples include entire cervices that have been resected during hysterectomy procedures, and cone biopsy samples, wherein the entire transformation zone of a lesion is removed from the cervix.

These tests will preferably be carried out in accordance with a set protocol. Samples will be analyzed within two hours following the extraction from the donor, or samples will be refrigerated in a buffer solution such as Hank's Balanced Salt Solution (HBSS) until analysis.

Once ready for testing, the tissue will be laid out in a Petri dish. A grid will be demarcated on it for ease of geometric registration, using sutures or black India Ink. The tissue will then be coated in acetic acid, which is known to enhance spectroscopic markers [POGU01]. Immediately following coating, the system will take measurements; acetic acid may be reapplied during testing as necessary. Standard pathological evaluation will be used to validate the findings of the invention.

7.4 In Vivo Tests in Human Subjects

Embodiments of the invention can also be evaluated by in vivo tests on subjects in a clinical setting. These experiments typically will have the following procedures (in the order listed): standard, white-light colposcopy; colposcopy enhanced with the invention; and histological evaluation through specimen biopsy.

8 Literature Cited

The entire disclosure of each of the following references is incorporated herein by reference.
[ACS04] American Cancer Society, Cancer Facts and Figures 2004.
[ALI04] J H Ali, Wang W B, Zevallos M, Alfano R R. “Near Infrared Spectroscopy and Imaging to Probe Differences in Water Content in Normal and Cancer Human Prostate Tissues,” Technology in Cancer Research and Treatment, 3(5): 491-497, 2004.
[BENA03] J M Benavides, Chang S, Park S Y, Richards-Kortum R, Mackinnon N, MacAulay C, Milboume A, Malpica A, Follen M. “Multispectral digital coloscopy for in vivo detection of cervical cancer.” Optics Express, 11(10): 1223-1236, 2003.
[BUXT91] E J Buxton, Luesley D M, Shafi M I, Rollason M. “Colposcopically directed punch biopsy: A potentially misleading investigation,” British Journal of Obstetrics and Gynecology, (98):1273-1276, 1991.
[CANT98] S B Cantor, Mitchell M F, Tortolero-Luna G, Bratka C S, Bodurka D C, Richards-Kortum R. “Cost-effectiveness analysis of diagnosis and management of cervical squamous intraepithelial lesions,” Obstetrics and Gynecology, 91 (2):270-7, 1998.
[CHIR98] L Chiriboga, Xie P, Yee H, Zarou D, Zakim D, Diem M. “Infrared Spectroscopy of Human Tissue IV. Detection of Dysplastic and Neoplastic Changes of Human Cervical Tissue Via Infrared Microscopy,” Cellular and Molecular Biology, 44(1):219-229, 1998.
[FAHE95] M T Fahey, Irwig L, Macaskill P. “Meta-analysis of Pap test accuracy,” American Journal of Epidemiology, 141 (7):680-689, 1995.
[GEOR02] I Georgakoudi, Sheets E E, Müller M G, Backman V, Crum C P, Badizadegan K, Dasari R R, Feld M S. “Trimodal Spectroscopy for the Detection and Characterization of Cervical Precancers in Vivo,” American Journal of Obstetric Gynecology, 186(3):374-382,2002.
[HORN99] R Hornung, Pham T H, Keefe K A, Berns M W, Tadir Y, Tromberg B J. “Quantitative Near-Infrared Spectroscopy of Cervical Dysplasia in Vivo,” Human Reproduction, 14(11):2908-2916, 1999.
[MIRA02] Y N Mirabal, Chang S K, Atkinson E N, Malpica A, Follen M, Richards-Kortum R. “Reflectance Spectroscopy for In Vivo Detection of Cervical Precancer,” Journal of Biomedical Optics, 7(4):587-594, 2002.
[MITC98] M F Mitchell, Schottenfeld D, Tortolero-Luna G, Cantor S B, Richards-Kortum R. “Colposcopy for the diagnosis of squamous intraepithelial lesions: a meta-analysis,” Obstetrics & Gynecology, 91:(626-631), 1998.
[NCI04] National Cancer Institute, Cancer Facts and Figures 2004.
[NORD01] Nordstrom R, L Burke, J M Niloff, J F Myrtle. “Identification of Cervical Intraepithelial Neoplasia (CIN) Using UV-Excited Fluorescence and Diffuse-Reflectance Tissue Spectroscopy,” Lasers in Surgery and Medicine, 29:118-127, 2001.
[POGU01] B Pogue, H B Kaufman, A Zelenchuk, W Harper, G C Burke, E E Burke, D M Harper. “Analysis of acetic acid-induced whitening of high-grade squamous intraepithelial lesions,” Journal of Biomedical Optics, 6(4):397-403, 2001.
[VANS91] van Staveren H J, C J M Moes, J van Merle, M J C van Gemert, S A Prahl. “Light scattering in Intralipid-10 in the wavelength range of 400-1100 nm,”Applied Optics, 30(31): 4507-4514, 1991.

What is claimed is:

1. An apparatus for optically analyzing a substrate, the apparatus comprising:
(a) a light source for directing light onto the substrate;
(b) optics for creating an optical path from light reflected from the substrate;
(c) a multiple wavelength imaging optical subsystem positioned in the optical path and comprising:
(i) one or more filters which are capable of one or both of:
(1) being alternatively or sequentially interposed in the optical path to extract one or more of wavelengths or wavelength bands of interest; or
(2) having their wavelength selectivity adjusted to extract one or more wavelengths or wavelength bands of interest; and
(ii) one or more imaging devices positioned to image the extracted wavelengths or wavelength bands of interest from the one or more filters; and
(d) an imaging device positioned in the optical path.

2. The apparatus of claim 1 further comprising a means for transmitting image data from the one or more imaging devices, which means is capable of being electronically coupled to a system to permit transmission of data from the imaging device to the system.

3. An optical scope comprising the apparatus of claim 1, wherein the optics are configured to permit a user to view the substrate via the optics.

4. The optical scope of claim 3 wherein the optical scope is one or both of:
(a) configured to permit a user to view an organ or anatomical region selected from the group consisting of airway, bronchi, vagina, cervix, uterus, urinary tract, bladder, esophagus, stomach, duodenum, rectum, sigmoid colon, colon, abdominal cavity, pelvic cavity, thoracic cavity, epidermis; and combinations thereof; or
(b) configured as a medical scope selected from the group consisting of bronchoscope, colonoscope, colposcope, cystoscope, hysteroscope, esophagogastroduodenoscope, laparoscope, proctosigmoidoscope, thorascope, and combinations thereof.

5. The optical scope of claim 3 wherein the optical scope is configured as a colposcope.

6. The optical scope of claim 3 wherein:
(a) the substrate comprises tissue; and
(b) the optical scope is configured to capture a full-frame image of an area of the tissue to be examined.

7. The optical scope of claim 6, wherein:
(a) the full-frame image comprises a number of pixels between about 4,000 and about 16,000,000; and
(b) the area to be examined is between 2 mm and 80 mm at its widest cross-section.

8. A system comprising the optical scope of claim 3 electronically coupled to a computer system, wherein the computer system comprises:
(a) a computer processor;
(b) a means for transmitting image data from the one or more imaging devices to the computer processor;
(c) an input device electronically coupled to the computer processor; and
(d) an output device electronically coupled to the computer processor.

9. The system of claim 8 wherein the processor is programmed and configured to permit the user to control one or more system capabilities selected from the group consisting of:
(a) electronically storing data, electronically transmitting data, or both from the images;
(b) viewing analytical results via an eyepiece user interface, an user console, or both;
(c) selecting an operating mode selected from the group consisting of:
(i) continuous-processing mode, thereby acquiring new sets of imagery on which to perform diagnostic analysis in a continuous, uninterrupted manner; and
(ii) single-frame processing mode, in which the user triggers the acquisition and analysis of a single set of images; and
(iii) combinations thereof;
and
(d) combinations thereof.

10. The system of claim 8 wherein the substrate is analyzed based on data from the images about the optical properties of the substrate by measuring the change in the intensity of reflected light over a predetermined spectral range, wherein:
(a) properties within a normal range are indicative of normal tissue;
(b) properties outside a normal range are indicative of abnormal tissue; and
(c) properties outside a normal range and in a recognized range for a class, species, or both of lesion are indicative of a lesion in said class, said species, or both.

11. The system of claim 8 wherein:
(a) the substrate comprises tissue,
(b) the computer system is programmed to conduct analysis of the image data from a full-frame image of the tissue to be analyzed from the one or more imaging devices.

12. The system of claim 11 wherein the optical scope:
(a) is configured as a colposcope; and
(b) is configured to capture a full-frame image of the cervix.

13. The system of claim 11 wherein the optical scope:
(a) is configured as a colposcope; and
(b) is configured to capture a full-frame image of the cervix wherein the image has from about 4,000 to about 16,000,000 pixels; and
(c) the system is programmed to analyze data from the image pixels.

14. The apparatus of claim I wherein the wavelengths of interest comprise one or more of individual wavelengths, combinations of individual wavelengths, or wavelength bands from one or more of the visible, near-infrared and infrared ranges.

15. The apparatus of claim 1 wherein the light source is filtered for removal of wavelengths selected from the group consisting of:
(a) wavelengths that cause image corruption,
(b) wavelengths that cause undesirable thermal effects in the images; and
(c) wavelengths that cause patient discomfort; and
(d) combinations thereof.

16. The apparatus of claim 1 wherein the light source is supplemented with additional light in one or more wavelengths of interest.

17. The apparatus of claim 1 wherein the one or more filters of the multiple wavelength imaging optical subsystem comprise 1, 2, 3, 4, 5, 6 or more filters selected from the group consisting of interference filters, dichroic filters, multiple-wavelength filter, and band-pass filters, and combinations thereof.

18. The apparatus of claim 17 wherein the one or more imaging devices of the multiple wavelength imaging optical subsystem comprise 1, 2, 3, 4, 5, 6 or more imaging devices, each corresponding to the one or more filters and each of which images a set of one or more continuous or discrete wavelengths or wavelength bands from one or both of the visible or near-infrared ranges.

19. The apparatus of claim 17 comprising 2, 3, 4, 5, 6 or more filters ordered in a series, wherein each filter in the series:
(a) permits a pre-selected set of one or more continuous or discrete wavelengths or wavelength bands to pass through and into an optical path that is directed to and imaged by an imaging device;
(b) reflects light that does not pass through the filter to a next filter in the series; and
(c) functions (a) and (b) are performed by all filters in the series in succession until a final filter, which reflects substantially any remaining light to an absorbent substrate.

20. The apparatus of claim 1 wherein the one or more imaging devices comprise an imaging device or imaging devices that simultaneously image a set of one or more continuous or discrete wavelengths or wavelength bands selected for spectrally distinctive behavior when interacting with the physical or chemical components of a tissue abnormality.

21. The apparatus of claim 20 wherein the one or more continuous or discrete wavelengths or wavelength bands are selected from one or more of the visible, near-infrared, and infrared ranges.

22. The apparatus of claim I wherein the one or more imaging devices comprise one or more of a CCD-based camera, a CMOS-based camera, an InGaAs-based camera, image intensifier tubes, or mechanically scanning mirror directed to a detector that receives sequentially scanned pixels to form a 2D image.

23. The apparatus of claim 1 wherein the one or more imaging devices do not comprise a point-source detector.

24. The optical scope of claim 3 wherein:
(a) the optics comprise a mechanism for splitting light in the optical path into two or more output optical paths;
(b) one of said output paths is directed via the optics to the imaging device for recording imagery; and
(c) another of said output paths is directed to an eyepiece for viewing by a user.

25. The optical scope of claim 24 further comprising an image display device, viewable by the user, which is electronically coupled to the imaging device.

26. The apparatus of claim 25 wherein the imaging device has a minimum resolution of 300,000 pixels.

27. The apparatus of claim 25 wherein the image display device is placed in an optical path leading to an eyepiece of the optical scope so that the user is able to view an image displayed on the image display device through the eyepiece.

28. The optical scope of claim 3 wherein the optics further comprise one or more of the following optical components:
(a) a mechanism for alternatively inserting one or more mirrors and beam- splitters into the optical path, such that:
(i) when one or more of the mirrors or beam-splitters are inserted into the optical path, the optical path is separated into at least two separate optical paths comprising:
(1) a first optical path directed to the multiple wavelength imaging optical subsystem; and
(2) a second optical path directed through the optics of the system, at least a portion of which reaches an eyepiece for viewing of the image by a user; and
(ii) when another one or more of the mirror(s) or beam-splitter(s) are inserted into the optical path, the multiple wavelength imaging optical subsystem is avoided, and the optical scope functions as a conventional scope;
(b) an electronically-alterable reflective-transmissive device, the properties of which can be changed based on an input signal to alternatively:
(i) separate the optical path into two separate optical paths:
(1) a first optical path directed to a multiple wavelength imaging optical subsystem; and
(2) a second optical directed through the remaining optics of the system, at least a portion of which reaches an eyepiece for viewing of the image by a user;
(ii) reflect the light to avoid the multiple wavelength imaging optical subsystem such that the optical scope functions as a conventional scope.

29. The apparatus of claim 28 wherein the optical components direct substantially all of the light that is NIR and IR light into the multiple wavelength imaging optical subsystem.

30. A system comprising the optical scope of claim 6 electronically coupled to a computer system, wherein the computer system comprises:
(a) a computer processor;
(b) a means for transmitting image data from the one or more imaging devices to the computer processor; and
(c) one or more peripherals electronically coupled to the computer processor, the one or more peripherals comprising:
(i) an input device; and
(ii) an output device.

31. The system of claim 30, wherein:
(a) the multiple wavelength imaging optical subsystem is configured to simultaneously image multiple images of the tissue;
(b) each image has a separate set of one or more continuous or discrete wavelengths or wavelength bands; and
(c) the computer system is programmed to analyze the images to identify spectral abnormalities to identify tissue abnormalities.

32. The system of claim 30, wherein:
(a) the multiple wavelength imaging optical subsystem is configured to image multiple images of the tissue;
(b) each image has a separate set of one or more continuous or discrete wavelengths or wavelength bands; and
(c) the computer system is programmed:
(i) to analyze the images to identify spectral abnormalities to identify one or more tissue abnormalities; and
(ii) to provide output to a user where the output is selected from the group consisting of:
(1) indicating a diagnosis of the one or more tissue abnormalities;
(2) classifying the one or more tissue abnormalities;
(3) ruling out one or more diagnoses or classes of abnormalities; and
(4) identifying the location of the one or more tissue abnormalities; and
(5) combinations thereof.

33. The system of claim 30 wherein the processor is programmed to identify variations in spectral signatures across a series of images from the imaging devices.

34. The system of claim 33 wherein one or more of the variations in spectral signatures are identified in light reflected from epithelial tissue of one or both of the cervix or the colon.

35. The system of claim 30 wherein the processor is programmed to analyze the substrate based on information from the images about one or more of the scattering, absorbing and other such optical properties of the substrate by measuring the change in the intensity of reflected light over a predetermined spectral range, and wherein:
(a) a change in the intensity of reflected light over a predetermined spectral range that is outside the range of the scattering, absorbing and other such optical properties of normal tissue represents a potential abnormality; or
(b) a change in the intensity of reflected light over a predetermined spectral range that is outside the range of the scattering, absorbing and other such optical properties for normal tissue and inside the range of the scattering and absorbing and other such optical properties of a tissue abnormality or class of tissue abnormalities represents potential abnormality or potential member of a class of abnormalities, or
(c) both.

36. The system of claim 30 further comprising a utility programmed:
(a) to extract subsections of said substrate wherein one or both of excessive light intensity or insufficient light intensity prevents imaging of said substrate with sufficient quality to permit the desired analysis, or
(b) to omit said subsections from diagnostic processing, or
(c) both.

37. The system of claim 30 further comprising a utility programmed:
(a) to identify spectral attributes in image sub-areas characteristic to a tissue abnormality or not characteristic of normal tissue; and
(b) to provide output to a user indicating the location of such image sub-areas.

38. The system of claim 37 wherein the output is selected from one or both of:
(a) a visible monochromatic or color image of said substrate displayed on a user interface; or
(b) one or more of the following displayed on the user interface:
(i) one or more indicators pointing out, circumscribing or highlighting any image sub-areas having spectral attributes characteristic of a tissue abnormality or not characteristic of normal tissue;
(ii) textual or symbolic information displayed on the user interface communicating information relating to classifying the tissue abnormality; or
(iii) textual or symbolic information communicating information of relevance to diagnosis or treatment of the tissue abnormality.

39. The system of claim 37 programmed to permit a user to provide input causing the system to provide an output image of the substrate:
(a) which is digitally or optically magnified;
(b) showing an individual wavelength or wavelength band; or
(c) showing raw spectral data from the substrate; or
(d) combinations thereof.

40. A method of detecting a tissue abnormality using the apparatus of claim 3, the method comprising:
(a) emitting light from the light source onto tissue;
(b) directing light emitted reflected from the tissue via the optics to the multiple wavelength imaging optical subsystem, and isolating one or more wavelengths or wavelength bands of interest;
(c) directing the one or more wavelengths or wavelength bands of interest to the one or more imaging devices, and using the imaging devices to record images of the one or more wavelengths or wavelength bands of interest;
(d) transferring image data from the images to a computational system; and
(e) analyzing the images for one or more spectral patterns associated with one or more tissue abnormalities.
